(19) United States
(12) Patent Application Publication
(10) Pub. No.: US 2026/0125360 A1
(43) Pub. Date: May 7, 2026

(54) HERBICIDAL IMIDAZOLE COMPOUNDS

(71) Applicant: SYNGENTA CROP PROTECTION AG, Basel (CH)

(72) Inventors: Suzanna DALE, Bracknell, Berkshire (GB); Philip Michael ELVES, Bracknell, Berkshire (GB); James Alan MORRIS, Bracknell, Berkshire (GB); Vikas SIKERVAR, Bracknell, Berkshire (GB); Samuel Vaughan WATKIN, Bracknell, Berkshire (GB)

(73) Assignee: SYNGENTA CROP PROTECTION AG, Basel (CH)

(21) Appl. No.: 19/117,705

(22) PCT Filed: Sep. 29, 2023
(86) PCT No.: PCT/EP2023/077015

§ 371 Date: Apr. 2, 2025

(30) Foreign Application Priority Data
Oct. 6, 2022 (IN) 202211057256

(51) Int. Cl. C07D 401/14; A01N 43/50; A01N 43/58; A01N 43/653; A01N 43/713; A01N 43/76; A01N 43/78; A01N 43/80; A01N 43/82; A01P 13/02; C07D 403/06; C07D 405/14; C07D 409/06; C07D 413/06; C07D 413/14; C07D 417/06; C07D 417/14

(52) U.S. Cl. CPC C07D 401/14; A01N 43/50; A01N 43/58; A01N 43/653; A01N 43/713; A01N 43/76; A01N 43/78; A01N 43/80; A01N 43/82; A01P 13/02; C07D 403/06; C07D 405/14; C07D 409/06; C07D 413/06; C07D 413/14; C07D 417/06; C07D 417/14

(57) ABSTRACT

The present invention relates to compounds of Formula (I), (I) or an agronomically acceptable salt of said compounds wherein Q, R², R³ and U are as defined herein. The invention further relates to herbicidal compositions which comprise a compound of Formula (I) and to the use of compounds of Formula (I) for controlling weeds, in particular in crops of useful plants.

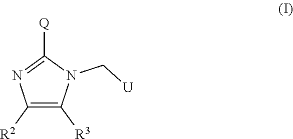

The present invention relates to herbicidal compounds, to herbicidal compositions which comprise the compounds, and to their use for controlling weeds, in particular in crops of useful plants, or for inhibiting plant growth.

WO2023/066783 discloses herbicidal imidazole compounds which feature a pyrimidinyl group corresponding to the U position identified in Formula (I) below. The compounds of the present invention are differentiated in that they contain a 5-membered heteroaryl at this position.

Thus, according to the present invention there is provided a compound of Formula (I):

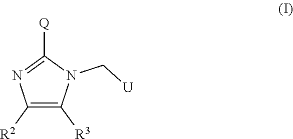

or an agronomically acceptable salt thereof,
wherein
Q is phenyl or a C-linked 6-membered heteroaryl wherein said phenyl or 6-membered heteroaryl is optionally substituted by one or more independent R¹;
U is a 5-membered heteroaryl optionally substituted by one or more independent R⁷;
R¹ is selected from the group consisting of halogen, C₁-C₄ alkyl, C₁-C₄ haloalkyl, C₁-C₄ alkoxy, C₁-C₄ haloalkoxy, C₃-C₆cycloalkyl, C₁-C₄alkoxyC₁-C₃alkyl-, C₁-C₄alkoxyC₁-C₃alkoxy-, C₁-C₄alkoxyC₁-C₄alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁴R⁵;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, —S(O)ₚC₁-C₄alkyl, —C(R⁶)═NOR⁸ and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁴R⁵;
R⁴ is selected from the group consisting of hydrogen, C₃-C₄cycloalkyl, C₁-C₄alkyl and C₁-C₄haloalkyl;
R⁵ is selected from the group consisting of hydrogen, C₃-C₄cycloalkyl, C₁-C₄alkyl and C₁-C₄haloalkyl;
R⁶ is hydrogen or C₁-C₂ alkyl;
R⁷ is independently of halogen, C₁-C₄ alkyl, C₃-C₄cycloalkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, —CN, C₂-C₄alkenyl, C₂-C₄alkynyl, —S(O)ₚC₁-C₄alkyl, —S(O)ₚC₁-C₄haloalkyl, —C(O)OC₁-C₄alkyl, —C(O)NR⁹R¹⁰, —NR¹¹COR¹² and —S(O)ₚNR¹³R¹⁴;
R⁸ is hydrogen or C₁-C₂ alkyl;
R⁹ is selected from the group consisting of hydrogen, C₃-C₄cycloalkyl, C₁-C₄alkyl and C₁-C₄haloalkyl;
R¹⁰ is selected from the group consisting of hydrogen, C₃-C₄cycloalkyl, C₁-C₄alkyl and C₁-C₄haloalkyl;
R¹¹ is selected from the group consisting of hydrogen, C₃-C₄cycloalkyl, C₁-C₄alkyl and C₁-C₄haloalkyl;
R¹² is selected from the group consisting of hydrogen, C₃-C₄cycloalkyl, C₁-C₄alkyl and C₁-C₄haloalkyl;
R¹³ is selected from the group consisting of hydrogen, C₃-C₄cycloalkyl, C₁-C₄alkyl and C₁-C₄haloalkyl;
R¹⁴ is selected from the group consisting of hydrogen, C₃-C₄cycloalkyl, C₁-C₄alkyl and C₁-C₄haloalkyl; and
p=0, 1 or 2.
C₁-C₄alkyl- and C₁-C₆alkyl-includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).
C₂-C₄alkenyl- includes, for example, —CH═CH₂ (vinyl) and —CH₂—CH═CH₂ (allyl).
C₂-C₄alkynyl- refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

Halogen (or halo) includes, for example, fluorine, chlorine, bromine or iodine. The same correspondingly applies to halogen in the context of other definitions, such as haloalkyl.

C₁-C₄haloalkyl-includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl and heptafluoro-n-propyl. C₁-C₂haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₆alkoxy includes methoxy and ethoxy.

C₁-C₄haloalkoxy- includes, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₁-C₄alkoxyC₁-C₃alkyl- includes, for example, methoxymethyl-.

C₁-C₄alkoxyC₁-C₃alkoxy- includes, for example, methoxyethoxy-.

C₁-C₄alkoxyC₁-C₃alkoxyC₁-C₃alkyl- includes, for example, meth-oxyethoxymethyl-.

C₃-C₆cycloalkyl includes cyclopropyl, cyclopentyl and cyclohexyl.

C₁-C₄alkyl-S— (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)— (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂— (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention, there is provided a compound of Formula (I), wherein U is selected from the group consisting of U1 to U46:

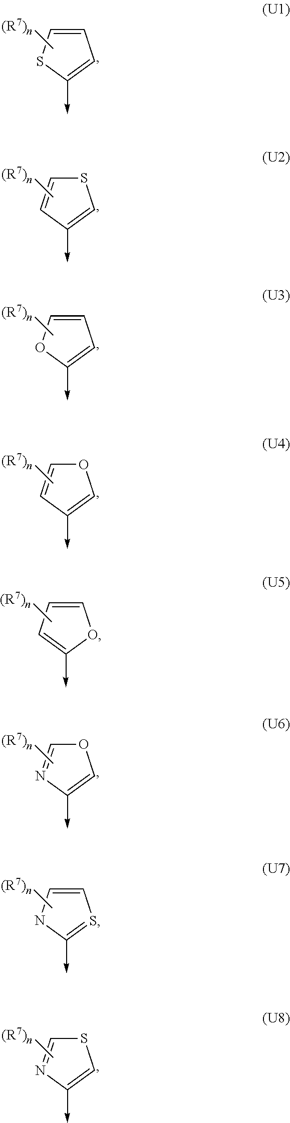

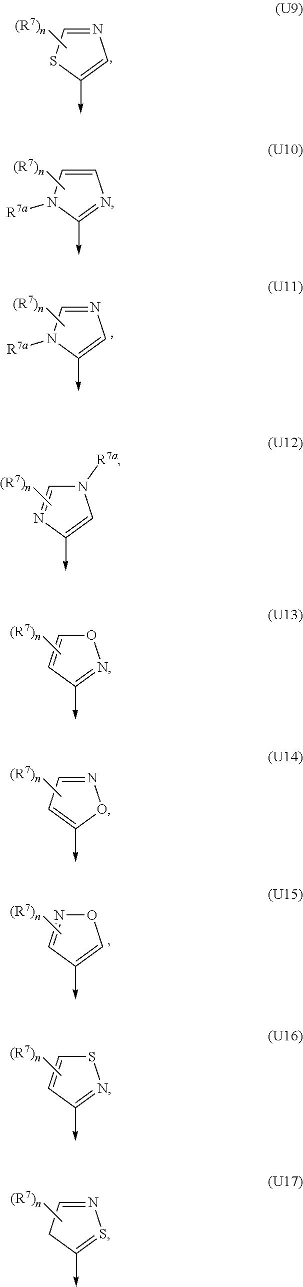

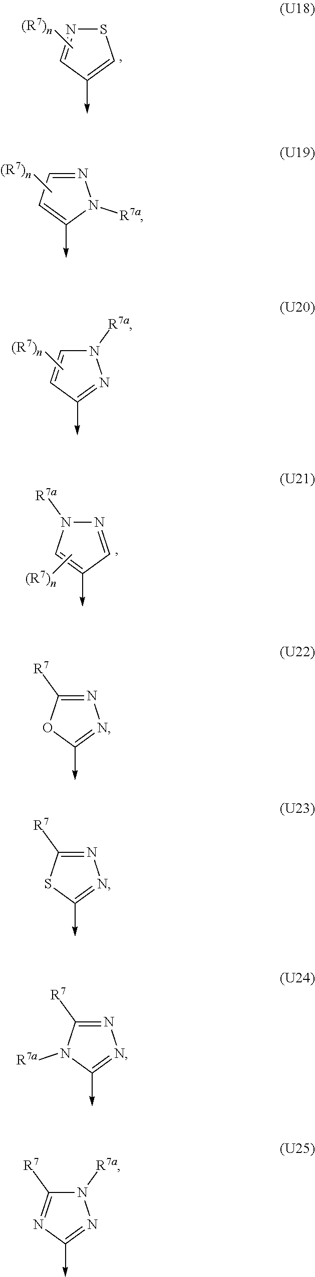

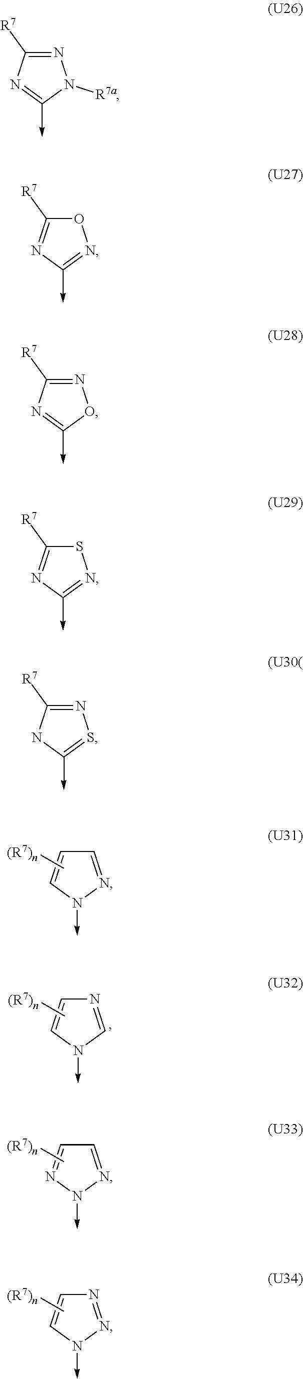

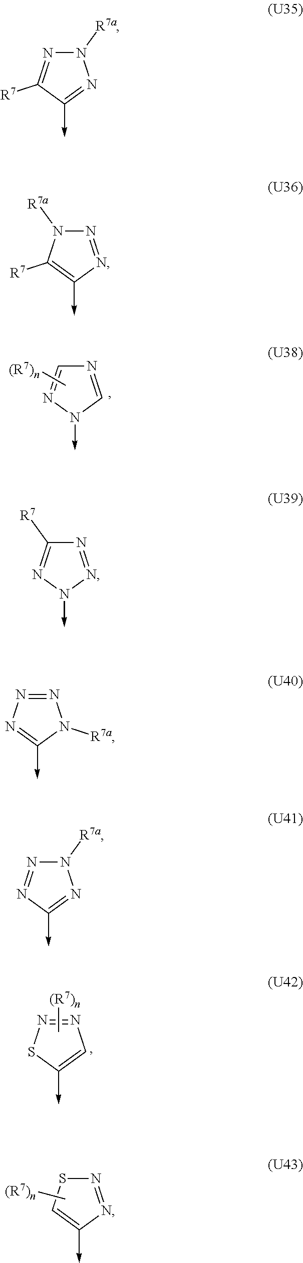

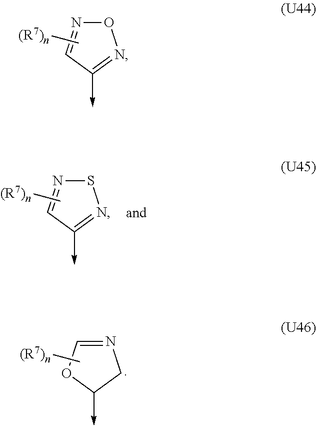

wherein
R⁷ is as defined in claim 1;
R^{7a} is selected from the group consisting of hydrogen, C₁-C₄alkyl, C₁-C₄haloalkyl and C₃-C₄cycloalkyl; and
n=0, 1 or 2.

In one embodiment of the present invention, U is selected from the group consisting of U5, U7, U13 U14, U16, U20, U23, U25, U27, U28, U36 and U41. In a more preferred embodiment of the present invention, U is selected from the group consisting of U13, U23, U25, U27 and U28, more preferably U25 and U27.

In another embodiment of the present invention, Q is preferably selected from the group consisting of Q 1 to Q-11:

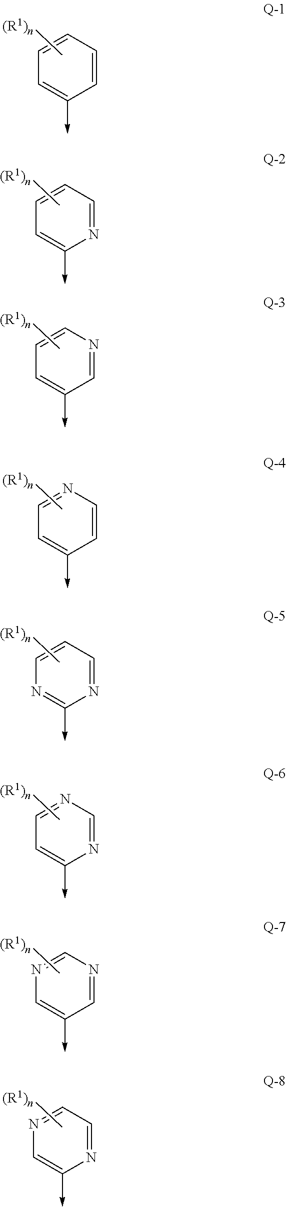

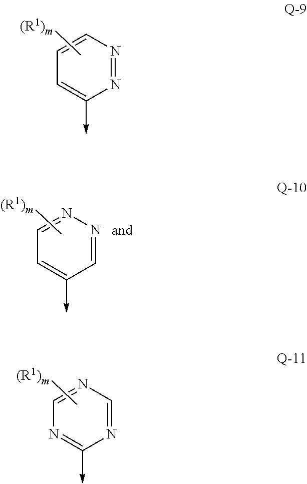

wherein R¹ is as defined in claim 1; and
mis 0, 1 or 2.

In a more preferred embodiment, Q is selected from the group consisting of Q-1, Q-3 and Q-7. In a particular embodiment of the invention, Q is Q-1.

Thus, in a particular embodiment of the present invention, the compound of Formula (I) is a compound of Formula (Ia):

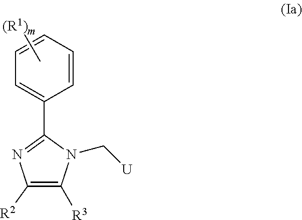

In another embodiment of the present invention, the compound of Formula (I) is selected from the group consisting of Formula (Iaa), (Iab), (Iac) and (Iad).

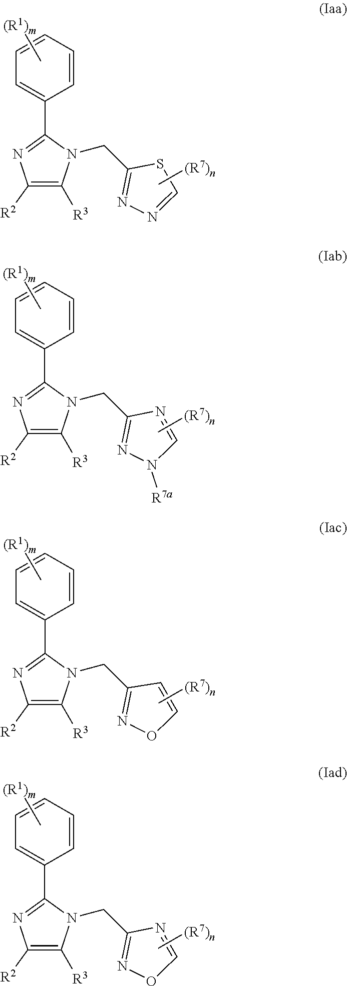

In a particular embodiment of the present invention there is provided a compound of Formula (I)—especially Formula (Ia), (Iaa), (Iab), (Iac) or (Iad) wherein m is 1, for example 4-CF₃.

In one embodiment of the present invention there is provided a compound of Formula (I), (Ia), (Iaa), (Iab), (Iac) or (Iad) wherein R² is halogen (e.g. Cl or Br) or C₁-C₄haloalkyl (e.g —CF₃, —CHF₂).

In one embodiment of the present invention there is provided a compound of Formula (I), (la), (Iaa), (Iab), (Iac) or (Iad), wherein, R³ is hydrogen or halogen (e.g Cl), particularly hydrogen.

Compounds of Formula (I) may contain asymmetric centres and may be present as a single enantiomer, pairs of enantiomers in any proportion or, where more than one asymmetric centre are present, contain diastereoisomers in all possible ratios. Typically, one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspoemulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen, flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1 (2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]-imidazolidine-2-one, 5-ethoxy-4-hydroxy-1-methyl-3-[4-(trifluoromethyl)-2-pyridyl]-imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl) pyrazol-3-yl]imidazolidin-2-one, (4R) 1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethyl-sulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonyl-methyl)-N-(5-methyl-1,3,4-oxadiazol-2-yl)-5-(trifluoromethyl)-[1,2,4]triazolo[4,3-a]-pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate, 6-chloro-4-(2,7-dimethyl-1-naphthyl)-5-hydroxy-2-methyl-pyridazin-3-one, tetrahydro-furan-2-ylmethyl (2R)-2-[(4-amino-3,5-dichloro-6-fluoro-2-pyridyl)oxy]-propanoate, (2R)-2-[(4-amino-3,5-dichloro-6-fluoro-2-pyridyl)oxy]propanoic acid, tetrahydrofuran-2-ylmethyl2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl6-amino-5-chloro-2-(4-chloro-2-fluoro-3-methoxyphenyl)-pyrimidine-4-carboxylate, 6-amino-5-chloro-2-(4-chloro-2-fluoro-3-methoxy-phenyl)-pyrimidine-4-carboxylic acid, 3-(3-chlorophenyl)-6-(5-hydroxy-1,3-dimethyl-pyrazole-4-carbonyl)-1,5-dimethyl-quinazoline-2,4-dione and [4-[3-(3-chlorophenyl)-1,5-dimethyl-2,4-dioxo-quinazoline-6-carbonyl]-2,5-dimethyl-pyrazol-3-yl]N,N-diethylcarbamate.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley soybean and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention can also be used in conjunction with crops that are tolerant to SDPS-inhibiting herbicides, such as those taught in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33BR (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

### PROCESSES FOR PREPARATION OF COMPOUNDS OF FORMULA (I)

Processes for preparation of compounds, e.g. a compound of formula (I) (which optionally can be an agrochemically acceptable salt thereof), are now described, and form further aspects of the present invention.

Compounds of formula I-1 is a compound of formula I wherein X¹ is a halogen (scheme 1). Compounds of formula I-2 are compounds of formula I wherein R³ is H (scheme 1).

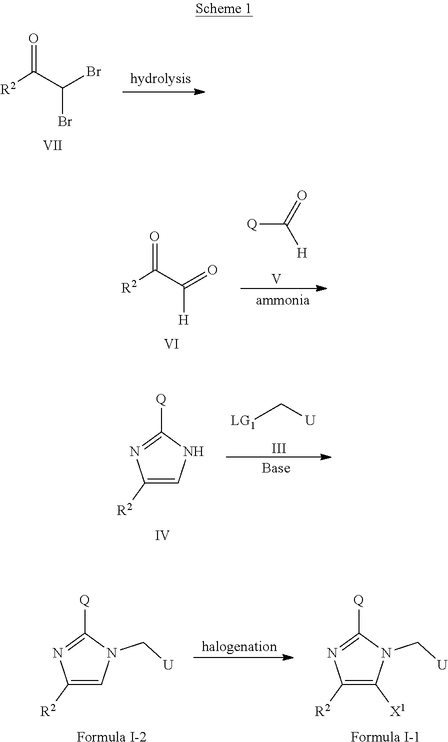

Compounds of formula I-1 can be prepared by a halogenation reaction, which involves for example, reacting compounds of formula I-2, wherein Q, R², and U are as defined in formula I, with halogenating reagents such as N-chlorosuccinimide (NCS), N-bromosuccinimide (NBS), or N-iodosuccinimide (NIS), optionally in the presence of an additive, such as for example p-toluenesulfonic acid. Alternatively, the halogenation may involve chlorine, bromine or iodine. Such halogenation reactions are carried out in a suitable solvent, such as chloroform, carbon tetrachloride, 1,2-dichloroethane, acetic acid, diethyl ether, acetonitrile or N,N-dimethylformamide, at temperatures between 20-200° C., preferably room temperature to 100° C.

Compounds of formula I-2, can be prepared by reacting compounds of formula IV, with reagents of the formula III, wherein U is as defined in formula I, and in which LG₁ is a halogen, preferably iodine, bromine or chlorine (or a pseudo-halogen leaving group, such as a (halo) alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, optionally in the presence of potassium iodide in an inert solvent such as tetrahydrofuran, dioxane, water, N, N-dimethylformamide DMF, N,N-dimethylacetamide or acetonitrile and the like, at temperatures between 0 and 120° C., by procedures well known to those skilled in the art.

Compounds of formula IV can be prepared by condensation reaction of compounds of formula VI or its hydrated form, wherein R² is as defined in formula I with compounds of formula V, in the presence of ammonia or its surrogates such as ammonium hydroxide. The reaction can be carried out in the presence of solvent such as methanol, tetrahydrofuran, ethanol, amongst others and at temperatures between 20-200° C., preferably room temperature to 100° C. Compounds of formula VI or its hydrated form, wherein R² is as defined in formula I can be prepared by hydrolysis of compounds of formula VII, wherein R² is as defined in formula I.

A compound of formula I-3 is a compound of Formula I, wherein R² is —CF₂H, R³ is —H and Q and U are as defined in formula I (scheme 2). Compounds of formula I-3 can be prepared from compounds of formula X via fluorination reactions using fluorinating reagents such as diethylaminosulfur trifluoride or bis(2-methoxyethyl) aminosulfur trifluoride amongst others (scheme 2). Compound of formula X can be prepared by reacting compounds of formula VIII with compounds of formula IX, following procedure analogous to as described in scheme 1 for the conversion of compounds of formula IV to compounds of formula I-2.

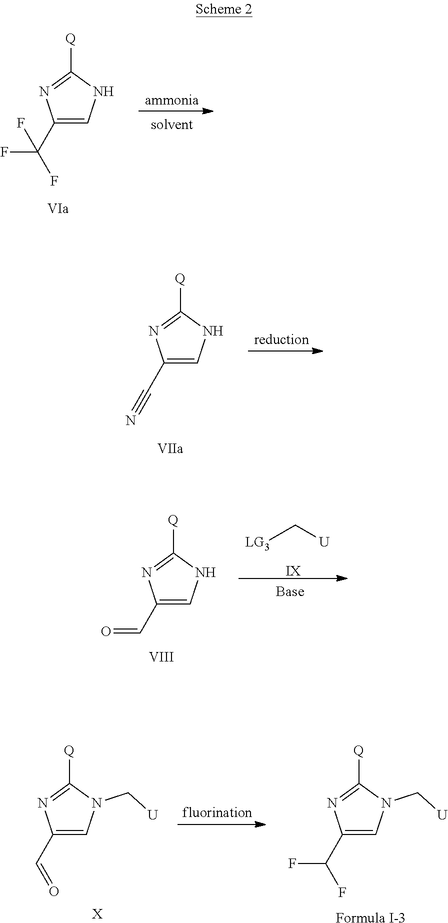

Compounds of formula VIII can be prepared by reacting compound of formula VIIa with a suitable reducing agent such as diisobutyl aluminium hydride. Compound of formula VIIa can be prepared by reacting compound of formula VIa with ammonium hydroxide or similar other ammonia surrogates to transform the trifluoromethyl group to a cyano group. Such reactions are well documented in the literature (see for example Matthews, D. P.; Whitten, J. P.; Mccarthy, J. R. J. Org. Chem. 1986, 51, 3228). Synthesis of imidazole compounds of Formula VIa are well documented in the literature (see for example Journal of Medicinal Chemistry, 2000, 43, 2165 and Synthetic Communications, 2020, 50, 700).

The following non-limiting examples provide specific synthesis methods for representative compounds of the present invention, as referred to in Tables 1a, 1b and 1c below.

### Example 1: Preparation of 1-methyl-3-[[4-(trifluoromethyl)-2-[4-(trifluoromethyl)phenyl]imidazol-1-yl]methyl]-1,2,4-triazole (1.003)

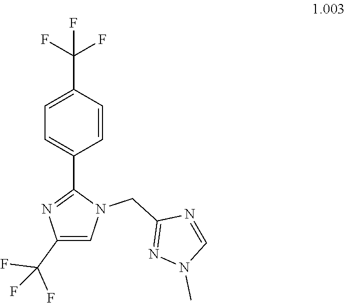 [SMILES: Cn1cnc(Cn2cc(C(F)(F)F)nc2-c2ccc(C(F)(F)F)cc2)n1]

### Step 1: Preparation of 1,1,1-trifluoro-3,3-dihydroxy-propan-2-one (I5)

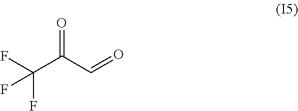

To a 250 mL flask equipped with a reflux condenser and thermometer was added sodium acetate (2.28 g, 27.6 mmol) and water (10.0 mL). 1,1-dibromo-3,3,3-trifluoroacetone (3.76 g, 1.90 mL, 13.2 mmol) was added to the above solution giving a turbid solution/suspension. The reaction mixture was heated to 100° C. for 30 minutes. The reaction mixture was then cooled to room temperature and used as such for the next step.

### Step 2: Preparation of 4-(trifluoromethyl)-2-[4-(trifluoromethyl)phenyl]-1H-imidazole (I6)

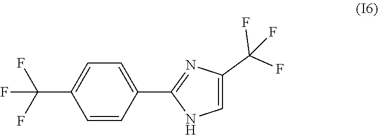 [SMILES: FC(F)(F)c1ccc(-c2nc(C(F)(F)F)c[nH]2)cc1]

To a 3-necked 250 mL flask was added 4-(trifluoromethyl)benzaldehyde (2.00 g, 1.57 mL, 11.0 mmol), methanol (40.0 mL) and 35% aq ammonia (8.8 g, 10.0 mL, 88 mmol, 8.0 equiv.) giving a colourless solution. To this was added the reaction mixture of I5 prepared in step 1 above slowly via dropping funnel dropwise over 60 mins. After complete addition the reaction was stirred at room temperature under air for 3 hours. The organics were removed under vacuum and the residue was partitioned between Ethyl acetate and Water and the phases separated. The aqueous phase was extracted with Ethyl acetate (3×20 mL). The organics were combined, washed with saturated brine and concentrated on to granulated celite. The crude material was subjected to column chromatography on silica gel using 0-50% ethyl acetate in cyclohexane to give 4-(trifluoromethyl)-2-[4-(trifluoromethyl)phenyl]-1H-imidazole 16 as a pale-yellow solid (2.57 g, 83% yield). 1H NMR (400 MHz, methanol) δ=8.10 (d, 2H), 7.80 (d, 2H), 7.75-7.71 (m, 1H).

### Step 3: Preparation of 1-methyl-3-[[4-(trifluoromethyl)-2-[4-(trifluoromethyl)phenyl]imidazol-1-yl]methyl]-1,2,4-triazole (1.003)

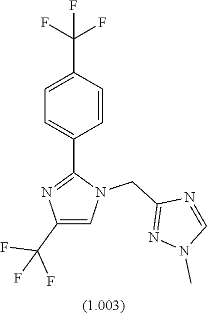 [SMILES: Cn1cnc(Cn2cc(C(F)(F)F)nc2-c2ccc(C(F)(F)F)cc2)n1]

To a 20 ml vial was added 4-(trifluoromethyl)-2-[4-(trifluoromethyl)phenyl]-1H-imidazole 16 (100.9 mg, 0.36 mmol) in acetonitrile (2 mL) followed by 3-(chloromethyl)-1-methyl-1,2,4-triazole; hydrochloride (99.4 mg, 0.60 mmol), potassium carbonate (125.6 mg, 0.91 mmol) and water (0.03 mL). The reaction mixture was shaken (500 RPM) for 22 h at 60° C. The reaction mixture was cooled to room temperature then the solvents were evaporated on a GeneVac HT-12. To the resulting material was added ethyl acetate (5 mL), shaken for 5 minutes, and then diluted with water (5 mL) and shaken for further 5 minutes. Organic layer was separated, and aqueous layer was extracted again with ethyl acetate (2×5 mL). Organic layers were combined and concentrated in a Genevac HT-12 to provide crude material, which was dissolved in 10% MeOH in DMSO (1.5 mL) and purified using reverse phase prep HPLC following solvent gradient method A described below to provide 1-methyl-3-[[4-(trifluoromethyl)-2-[4-(trifluoromethyl)phenyl]imidazol-1-yl]methyl]-1,2,4-triazole 1.003. ¹H NMR (500 MHz, chloroform) δ=8.05 (s, 1H), 8.00 (d, 2H), 7.77 (d, 2H), 7.57 (s, 1H), 5.22 (s, 2H), 3.95 (s, 3H).

Method A: Solvent A: Water with 0.05% Trifluoroacetic Acid (TFA); Solvent B: Acetonitrile with 0.05% (TFA)

| Time | Solvent A | Solvent B | Flow |
|---|---|---|---|
| (mins) | (%) | (%) | (ml/mn) |
| 0.00 | 60 | 40 | 33 |
| 1.50 | 60 | 40 | 33 |
| 1.51 | 40 | 60 | 33 |
| 7.0 | 20 | 80 | 33 |
| 7.3 | 0 | 100 | 33 |
| 9.2 | 0 | 100 | 33 |
| 9.8 | 95 | 5 | 33 |
| 11.35 | 95 | 5 | 33 |
| 11.40 | 95 | 5 | 3.5 |

### Example 2: Preparation of 2-chloro-5-[4-(difluoromethyl)-1-[(1-methyl-1,2,4-triazol-3-yl)methyl]imidazol-2-yl]-3-fluoro-pyridine (1.015)

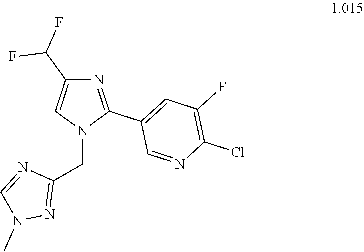 [SMILES: Cn1cnc(Cn2cc(C(F)F)nc2-c2cnc(Cl)c(F)c2)n1]

### Step 1: Preparation of 2-chloro-3-fluoro-5-[4-(trifluoromethyl)-1H-imidazol-2-yl]pyridine (I7)

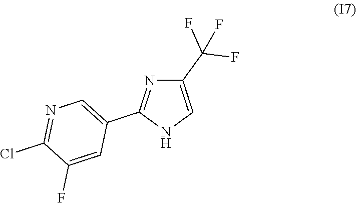 [SMILES: Fc1cc(-c2nc(C(F)(F)F)c[nH]2)cnc1Cl]

To a solution of 1,1-dibromo-3,3,3-trifluoroacetone (12.8 g, 45.12 mmol) in water (30 mL) was added sodium acetate (9.46 g, 112.82 mmol) resulting a turbid solution. This reaction mixture was heated to 100° C. for 30 minutes. The reaction mixture was cooled to room temperature. To this solution, were added 6-chloro-5-fluoro-pyridine-3-carbaldehyde (6 g, 37.60 mmol) in methanol (120 mL) followed by aqueous ammonia (30 mL) slowly. The reaction mixture was stirred at room temperature overnight. The reaction mixture was extracted with ethyl acetate (3×200 mL). The organic layer was washed with saturated bicarbonate solution (100 mL) and then with brine (100 mL), dried over anhydrous sodium sulphate, filtered, and concentrated to get the crude material. The crude material was subjected to column chromatography on silica gel using 0-50% ethyl acetate in cyclohexane to give 2-chloro-3-fluoro-5-[4-(trifluoromethyl)-1H-imidazol-2-yl]pyridine I7 as a yellow gummy mass (6 g, quant. yield). 1H NMR (400 MHz, CHLOROFORM-d) δ=12.83 (br s, 1H), 8.82 (d, 1H), 8.19 (dd, 1H), 7.39 (s, 1H).

### Step 2: Preparation of 2-(6-chloro-5-fluoro-3-pyridyl)-1H-imidazole-4-carbonitrile (I8)

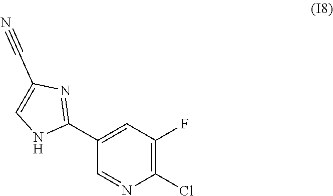 [SMILES: N#Cc1c[nH]c(-c2cnc(Cl)c(F)c2)n1]

To a single neck 250 mL flask, were added 2-chloro-3-fluoro-5-[4-(trifluoromethyl)-1H-imidazol-2-yl]pyridine (17, 6 g, 22.59 mmol), methanol (30 mL) and aqueous ammonia (54 mL) and the reaction mixture was stirred at 70° C. for 10 h. After this time, the reaction mixture was cooled to room temperature and diluted with water (150 mL). This mixture was then extracted with ethyl acetate (3×150 mL). The combined organic layer was washed with saturated bicarbonate solution (100 mL) and then with brine (100 mL), dried over anhydrous sodium sulphate, filtered, and concentrated to get the crude material. The crude material was purified by silica gel column chromatography using 0-50% ethyl acetate in cyclohexane to give 2-(6-chloro-5-fluoro-3-pyridyl)-1H-imidazole-4-carbonitrile I8 as gummy oil (4 g, 79% yield). 1H NMR (400 MHz, METHANOL-d4) δ=8.75 (d, 1H), 8.18 (dd, 1H) 8.06 (s, 1H).

### Step 3: Preparation of 2-(6-chloro-5-fluoro-3-pyridyl)-1H-imidazole-4-carbaldehyde (I9)

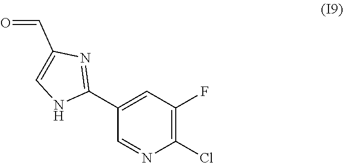 [SMILES: O=Cc1c[nH]c(-c2cnc(Cl)c(F)c2)n1]

In a 100 mL three neck flask, 2-(6-chloro-5-fluoro-3-pyridyl)-1H-imidazole-4-carbonitrile (I8, 0.5 g, 2.24 mmol) was dissolved in tetrahydrofuran (10 mL). This was cooled to −70° C. To this solution, was added di-isobutylaluminum hydride solution (1.0 M) in toluene (4.5 mL, 4.5 mmol), maintaining the temperature <−68° C. After the complete addition, this was stirred at −70° C. for 1 h, then slowly warmed to 0° C. The reaction mixture was stirred at 0° C. for 1 h and then quenched by slowly adding ˜5 mL ammonium chloride solution. The reaction mixture was then extracted with ethyl acetate (3×50 mL). The combined organic layer was concentrated to get the crude product which was purified by silica gel column chromatography using 30-40% ethyl acetate in cyclohexane. Upon concentration of the pure fractions, the 2-(6-chloro-5-fluoro-3-pyridyl)-1H-imidazole-4-carbaldehyde I9 was obtained as yellow brown solid (0.38 g, 75%). 1H NMR (400 MHz, METHANOL-d4) δ=9.81 (s, 1H), 8.82 (d, 1H), 8.26 (dd, 1H), 8.07 (s, 1H).

Step 4: Preparation of (1-methyl-1,2,4-triazol-3-yl)methyl methanesulfonate (I10)

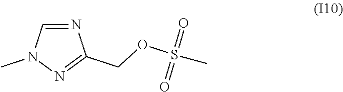

In a dry two neck 50 mL flask, (1-methyl-1,2,4-triazol-3-yl) methanol (1.5 g, 13 mmol) was dissolved in tetrahydrofuran and cooled to 0° C. To this, N, N-diethylethanamine (4 g, 40 mmol) was added. To this solution, 4-dimethylaminopyridine (1.6 g, 13 mmol) and methanesulfonyl chloride (4.6 g, 40 mmol) were added and the reaction mixture was stirred at room temperature for 16 h. The reaction mixture was quenched by slowly adding 50 mL of saturated sodium bicarbonate solution and then extracted with ethyl acetate (3×50 mL). The combined organic layer was dried over Sodium sulphate, filtered, and concentrated under reduced pressure to get off-brown liquid. The crude (1-methyl-1,2,4-triazol-3-yl)methyl methanesulfonate 110 was used directly for the next step (2 g, 79% yield). 1H NMR (400 MHz, CHLOROFORM-d) δ=8.12 (s, 1H), 4.62 (s, 2H), 3.92 (s, 3H), 3.67 (s, 3H).

### Step 5: Preparation of 2-(6-chloro-5-fluoro-3-pyridyl)-1-[(1-methyl-1,2,4-triazol-3-yl)methyl]imidazole-4-carbaldehyde (I11)

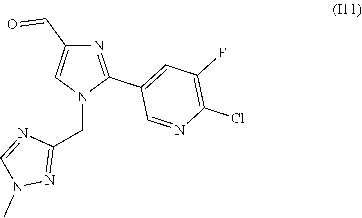 [SMILES: Cn1cnc(Cn2cc(C=O)nc2-c2cnc(Cl)c(F)c2)n1]

To a solution of 2-(6-chloro-5-fluoro-3-pyridyl)-1H-imidazole-4-carbaldehyde (I9, 1.7 g, 7.5 mmol) in acetonitrile (75 mL) was added potassium carbonate (2.6 g, 19 mmol) followed by potassium iodide (0.25 g, 1.5 mmol). The reaction mixture was heated to 60° C. A solution of (1-methyl-1,2,4-triazol-3-yl)methyl methanesulfonate (I10, 1.7 g, 9.0 mmol) in acetonitrile (10 mL) were added in the reaction mixture at this temperature and the stirring was continued at the same temperature for 16 h. After this time, the reaction mixture was cooled to room temperature and quenched by adding 100 mL of saturated sodium bicarbonate solution. The mixture was extracted with 10% methanol in ethyl acetate (3×200 mL). The combined organic layer was dried over sodium sulphate, filtered, and concentrated under reduced pressure to get the crude compound. The crude was purified by silica gel chromatography using 0-100% ethyl acetate in cyclohexane to yield 2-(6-chloro-5-fluoro-3-pyridyl)-1-[(1-methyl-1,2,4-triazol-3-yl)methyl]imidazole-4-carbaldehyde 111 (1.2 g, 49% Yield) as yellow solid. ¹H NMR (400 MHz, ACETONITRILE-d3) δ=9.80 (s, 1H), 8.63 (d, 1H), 8.18 (dd, 1H), 8.13 (s, 1H), 7.98 (s, 1H), 5.30 (s, 2H), 3.82 (s, 3H).

### Step 6: Preparation of 2-chloro-5-[4-(difluoromethyl)-1-[(1-methyl-1,2,4-triazol-3-yl)methyl]imidazol-2-yl]-3-fluoro-pyridine (1.015)

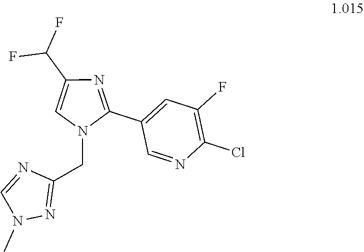 [SMILES: Cn1cnc(Cn2cc(C(F)F)nc2-c2cnc(Cl)c(F)c2)n1]

To a solution of 2-(6-chloro-5-fluoro-3-pyridyl)-1-[(1-methyl-1,2,4-triazol-3-yl)methyl]imidazole-4-carbaldehyde (I11, 0.30 g, 0.93 mmol) in dichloromethane (18 mL) at 0° C. was added diethylaminosulfur trifluoride (1.27 g, 7.48 mmol) dropwise. After the addition, the mixture was allowed to attain room temperature and stirred at this temperature for overnight. After this time, the reaction mixture was cooled to 0° C., slowly quenched with 10% saturated bicarbonate solution (˜50 mL). The layers were separated. The aqueous layer was extracted with ethyl acetate (3×100 mL), washed with brine solution (50 mL), dried over sodium sulphate, filtered, and concentrated to get the crude material. The crude was purified by silica gel chromatography using 0-100% ethyl acetate in cyclohexane to yield 2 2-chloro-5-[4-(difluoromethyl)-1-[(1-methyl-1,2,4-triazol-3-yl)methyl]imidazol-2-yl]-3-fluoro-pyridine 1.015 (0.165 g, 51% Yield) as off-white solid. 1H NMR (400 MHz, METHANOL-d4) δ=8.60 (d, 1H), 8.36 (s, 1H), 8.21 (dd, 1H), 7.67 (t, 1H), 6.75 (t, 1H), 5.36 (s, 2H), 3.90 (s, 3H).

### Example 3: Preparation of 2-chloro-5-[5-chloro-4-(difluoromethyl)-1-[(1-methyl-1,2,4-triazol-3-yl)methyl]imidazol-2-yl]-3-fluoro-pyridine (1.155)

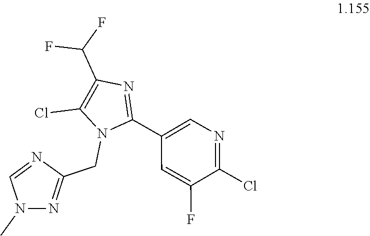 [SMILES: Cn1cnc(Cn2c(-c3cnc(Cl)c(F)c3)nc(C(F)F)c2Cl)n1]

To a 10 mL flask, 2-chloro-5-[4-(difluoromethyl)-1-[(1-methyl-1,2,4-triazol-3-yl)methyl]imidazol-2-yl]-3-fluoro-pyridine (1.015, 0.05 g, 0.14 mmol) was dissolved in acetonitrile (1 mL), and N-chlorosuccinimide (0.024 g, 0.18 mmol) was added to it. The reaction mixture was stirred at room temperature for 16 h. After this time, the reaction was quenched with 10% sodium thiosulphate solution (50 mL) and extracted with ethyl acetate (2×50 mL), dried over sodium sulphate, filtered, and concentrated to get the crude material. The crude material was purified by silica gel column chromatography using 80-100% ethyl acetate in cyclohexane. The white solid obtained after chromatography contain succinimide impurity. This was further purified by reverse phase using 40% acetonitrile in water to get 2-chloro-5-[5-chloro-4-(difluoromethyl)-1-[(1-methyl-1,2,4-triazol-3-yl)methyl]imidazol-2-yl]-3-fluoro-pyridine 1.155 as white solid (0.01 g, 18% yield). ¹H NMR (400 MHz, METHANOL-d4) δ=8.59 (d, 1H), 8.37 (s, 1H), 8.21 (dd, 1H), 6.82 (t, 1H), 5.38 (s, 2H), 3.90 (s, 3H).

### LCMS Method 1:

Spectra were recorded on a ACQUITY Mass Spectrometer from Waters Corporations (QDa or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 3.0 min; Flow (ml/min) 0.75.

### LCMS Method 2:

Spectra was recorded in either one of the instruments below:—

Agilent 1100 Series LC/MSD system with DAD\ELSD Alltech 2000ES and Agilent LC\MSD VL (G1956B), SL (G1956B) mass-spectrometer.

Agilent 1200 Series LC/MSD system with DAD\ELSD Alltech 3300 and Agilent LC\MSD G6130A, G6120B mass-spectrometer.

Agilent Technologies 1260 Infinity LC/MSD system with DAD\ELSD Alltech 3300 and Agilent LC\MSDG6120B mass-spectrometer.

Agilent Technologies 1260 Infinity II LC/MSD system with DAD\ELSD G7102A 1290 Infinity II and Agilent LC\MSD G6120B mass-spectrometer.

Agilent 1260 Series LC/MSD system with DAD\ELSD and Agilent LC\MSD (G6120B) mass-spectrometer.

UHPLC Agilent 1290 Series LC/MSD system with DAD\ELSD and Agilent LC\MSD (G6125B) mass-spectrometer.

Column: Agilent Poroshell 120 SB-C18 4.6×30 mm 2.7 μm

Column Temperature: 60° C., Mobile phase: A-water (0.1% formic acid), B-acetonitrile (0.1% formic acid)

Flow rate: 3 ml/min, Gradient: 0.01 min-1% B, 1.5 min-100% B, 1.73 min-100% B, MS Ionization mode: Electrospray ionization (ESI), MS Scan range: 83-600 m/z and with UV detection: 215 nm, 254 nm, 280 nm.

### LCMS Method 3:

Spectra was recorded in one of the instrument below:—

Agilent 1100 Series LC/MSD system with DAD\ELSD Alltech 2000ES and Agilent LC\MSD VL (G1956B), SL (G1956B) mass-spectrometer.

Agilent 1200 Series LC/MSD system with DAD\ELSD Alltech 3300 and Agilent LC\MSD G6130A, G6120B mass-spectrometer.

Agilent Technologies 1260 Infinity LC/MSD system with DAD\ELSD Alltech 3300 and

Agilent LC\MSDG6120B mass-spectrometer.

Agilent Technologies 1260 Infinity II LC/MSD system with DAD\ELSD G7102A 1290 Infinity II and Agilent LC\MSD G6120B mass-spectrometer.

Agilent 1260 Series LC/MSD system with DAD\ELSD and Agilent LC\MSD (G6120B) mass-spectrometer.

UHPLC Agilent 1290 Series LC/MSD system with DAD\ELSD and Agilent LC\MSD (G6125B) mass-spectrometer.

Column: Agilent Poroshell 120 SB-C18 4.6×30 mm 2.7 μm, Column Temperature: 60° C.

Mobile phase: A-water (0.1% formic acid), B-acetonitrile (0.1% formic acid), Flow rate: 1.5 ml/min, Gradient: 0.01 min-1% B, 5.00 min-100% B, 5.99 min-100% B, MS Ionization mode: Electrospray ionization (ESI), MS Scan range: 83-1000 m/z and with UV detection: 215 nm, 254 nm, 280 nm

TABLE 1a
Compounds of the Present Invention

| Compound | STRUCTURE | ¹H NMR (500 MHz, CDCl₃) |
|---|---|---|
| 1.001 | 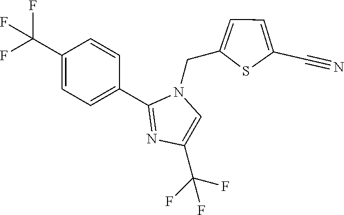 [SMILES: N#Cc1ccc(Cn2cc(C(F)(F)F)nc2-c2ccc(C(F)(F)F)cc2)s1] | δ = 7.77 (d, 2H), 7.70 (d, 2H), 7.54 (d, 1H), 7.43 (s, 1H), 6.90 (d, 1H), 5.41 (s, 2H) |
| 1.002 | 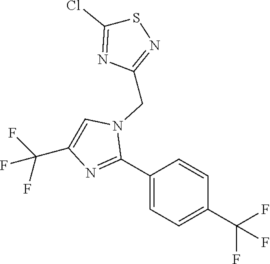 [SMILES: FC(F)(F)c1ccc(-c2nc(C(F)(F)F)cn2Cc2nsc(Cl)n2)cc1] | δ = 7.88 (d, 2H), 7.77 (d, 2H), 7.57 (d, 1H), 5.39 (s, 2H) |
| 1.003 | 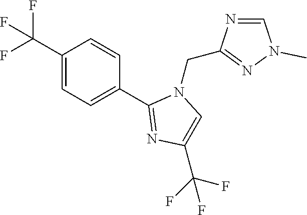 [SMILES: Cn1cnc(Cn2cc(C(F)(F)F)nc2-c2ccc(C(F)(F)F)cc2)n1] | δ = 8.05 (s, 1H), 8.00 (d, 2H), 7.77 (d, 2H), 7.57 (s, 1H), 5.22 (s, 2H), 3.95 (s, 3H) |
| 1.004 | 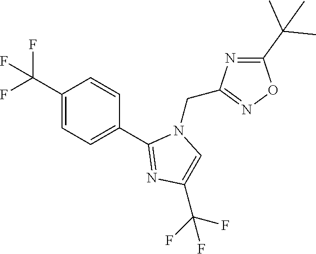 [SMILES: CC(C)(C)c1nc(Cn2cc(C(F)(F)F)nc2-c2ccc(C(F)(F)F)cc2)no1] | δ = 7.93 (d, 2H), 7.77 (d, 2H), 7.57 (s, 1H), 5.23 (s, 2H), 1.46 (s, 9H) |
| 1.005 | 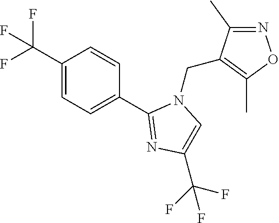 [SMILES: Cc1noc(C)c1Cn1cc(C(F)(F)F)nc1-c1ccc(C(F)(F)F)cc1] | δ = 7.93 (d, 2H), 7.77 (d, 2H), 7.57 (s, 1H), 5.23 (s, 2H), 1.46 (s, 6H) |
| 1.006 | 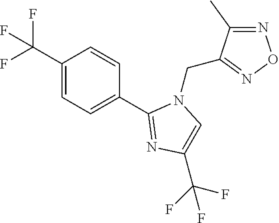 [SMILES: Cc1nonc1Cn1cc(C(F)(F)F)nc1-c1ccc(C(F)(F)F)cc1] | δ = 7.79 (d, 2H), 7.75 (d, 2H), 7.38 (s, 1H), 5.37 (s, 2H), 2.13 (s, 3H) |
| 1.007 | 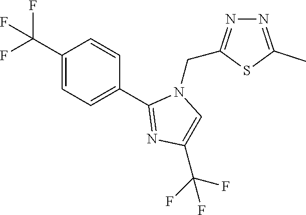 [SMILES: Cc1nnc(Cn2cc(C(F)(F)F)nc2-c2ccc(C(F)(F)F)cc2)s1] | δ = 7.81 (d, 2H), 7.78 (d, 2H), 7.51 (s, 1H), 5.58 (s, 2H), 2.80 (s, 3H) |
| 1.008 | 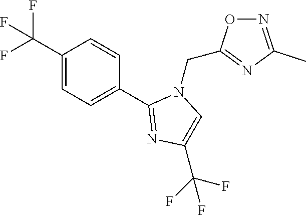 [SMILES: Cc1noc(Cn2cc(C(F)(F)F)nc2-c2ccc(C(F)(F)F)cc2)n1] | δ = 7.81 (d, 2H), 7.78 (d, 2H), 7.55 (s, 1H), 5.37 (s, 2H), 2.45 (s, 3H) |
| 1.009 | 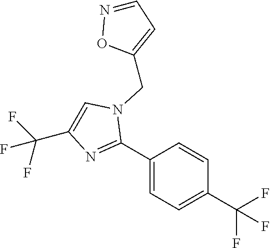 [SMILES: FC(F)(F)c1ccc(-c2nc(C(F)(F)F)cn2Cc2ccno2)cc1] | δ = 8.30-8.27 (m, 1H), 7.79-7.72 (m, 4H), 7.47 (s, 1H), 6.23-6.19 (m, 1H), 5.33 (s, 2H) |
| 1.010 | 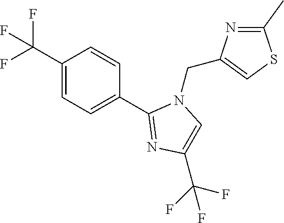 [SMILES: Cc1nc(Cn2cc(C(F)(F)F)nc2-c2ccc(C(F)(F)F)cc2)cs1] | δ = 7.86 (d, 2H), 7.74 (d, 2H), 7.47 (s, 1H), 6.91 (s, 1H), 5.23 (s, 2H), 2.73 (s, 3H) |
| 1.011 | 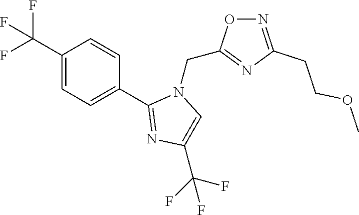 [SMILES: COCCc1noc(Cn2cc(C(F)(F)F)nc2-c2ccc(C(F)(F)F)cc2)n1] | δ = 7.82-7.75 (m, 4H), 7.56 (s, 1H), 5.38 (s, 2H), 3.77 (t, 2H), 3.36 (s, 3H), 3.04 (t, 2H) |
| 1.012 | 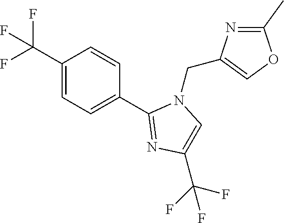 [SMILES: Cc1nc(Cn2cc(C(F)(F)F)nc2-c2ccc(C(F)(F)F)cc2)co1] | δ = 7.88 (d, 2H), 7.75 (d, 2H), 7.47 (s, 2H), 5.03 (s, 2H), 2.48 (s, 3H) |
| 1.013 | 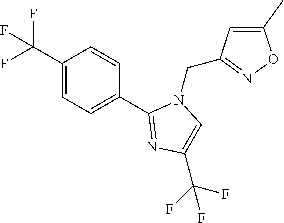 [SMILES: Cc1cc(Cn2cc(C(F)(F)F)nc2-c2ccc(C(F)(F)F)cc2)no1] | δ = 7.80-7.74 (m, 4H), 7.43 (s, 1H), 5.85 (s, 1H), 5.23 (s, 2H), 2.45 (s, 3H) |
| 1.014 | 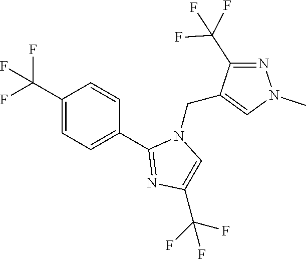 [SMILES: Cn1cc(Cn2cc(C(F)(F)F)nc2-c2ccc(C(F)(F)F)cc2)c(C(F)(F)F)n1] | δ = 7.75-7.68 (m, 4H), 7.34 (s, 1H), 7.13 (s, 1H), 5.20 (s, 2H), 3.93 (s, 3H) |
| 1.015 | 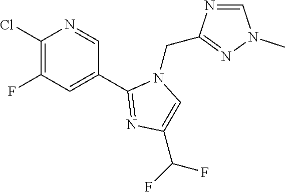 [SMILES: Cn1cnc(Cn2cc(C(F)F)nc2-c2cnc(Cl)c(F)c2)n1] | ¹H NMR (400 MHz, METH- ANOL-d4) δ = 8.60 (d, 1 H), 8.36 (s, 1 H), 8.21 (dd, 1 H), 7.67 (t, 1 H), 6.75 (t, 1 H), 5.36 (s, 2 H), 3.90 (s, 3 H) |
| 1.016 | 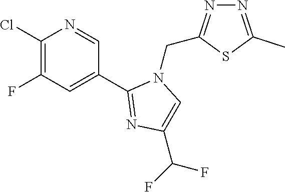 [SMILES: Cc1nnc(Cn2cc(C(F)F)nc2-c2cnc(Cl)c(F)c2)s1] | ¹H NMR (400 MHz, CHLORO- FORM-d) δ = 8.53 (d, 1 H), 7.94 (dd, 1 H) 7.44 (t, 1 H), 6.69 (t, 1 H), 5.59 (s, 2 H), 2.81 (s, 3 H) |
| 1.017 | 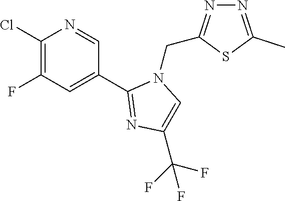 [SMILES: Cc1nnc(Cn2cc(C(F)(F)F)nc2-c2cnc(Cl)c(F)c2)s1] | ¹H NMR (400 MHz, CHLORO- FORM-d) δ = 8.54 (d, 1 H), 7.96 (dd, 1 H), 7.55 (d, 1 H), 5.60 (s, 2 H), 2.83 (s, 3 H) |
| 1.018 | 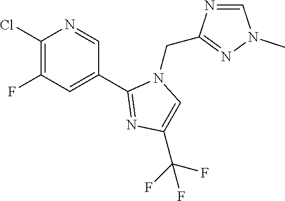 [SMILES: Cn1cnc(Cn2cc(C(F)(F)F)nc2-c2cnc(Cl)c(F)c2)n1] | ¹H NMR (400 MHz, chloroform-d) δ = 8.79 (d, 1H), 8.17 (dd, 1H), 8.05 (s, 1H), 7.59 (d, 1H), 5.23 (s, 2H), 3.95 (s, 3H) |
| 1.019 | 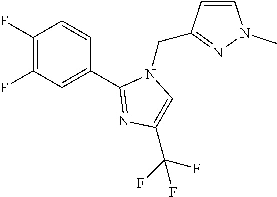 [SMILES: Cn1ccc(Cn2cc(C(F)(F)F)nc2-c2ccc(F)c(F)c2)n1] | ¹H NMR (400 MHz, CHLORO- FORM-d) δ =7.60 (m, 1 H), 7.47 (m, 1 H), 7.40 (m, 1 H), 7.36 (d, 1 H), 7.26 (m, 1H), 6.09 (d, 1 H), 5.14 (s, 2 H), 3.92 (s, 3 H) |
| 1.020 | 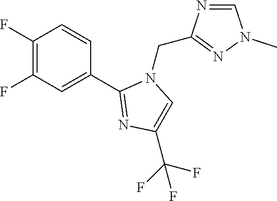 [SMILES: Cn1cnc(Cn2cc(C(F)(F)F)nc2-c2ccc(F)c(F)c2)n1] | ¹H NMR (400 MHz, chloroform-d) δ = 7.98 (s, 1H), 7.70 (dd, 1H), 7.57-7.51 (m, 1H), 7.45 (s, 1H), 7.26-7.24 (m, 1H), 5.12 (s, 2H), 3.88 (s, 3H) |
| 1.021 | 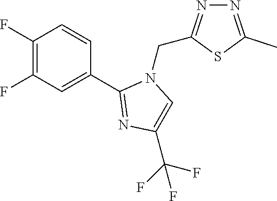 [SMILES: Cc1nnc(Cn2cc(C(F)(F)F)nc2-c2ccc(F)c(F)c2)s1] | ¹H NMR (400 MHz, CHLORO- FORM-d) δ = 7.56 (ddd, 1 H), 7.48 (q, 1 H), 7.40-7.44 (m, 1 H), 7.29- 7.35 (m, 1 H), 5.57 (s, 2 H), 2.82 (s, 3 H) |
| 1.022 | 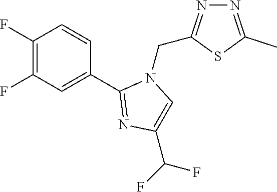 [SMILES: Cc1nnc(Cn2cc(C(F)F)nc2-c2ccc(F)c(F)c2)s1] | ¹H NMR (400 MHz, CHLORO- FORM-d) δ = 7.44-7.49 (m, 1 H), 7.30-7.34 (m, 2 H), 7.22-7.26 (m, 1 H), 6.69 (t, 1 H), 5.56 (s, 2 H), 2.80 (s, 3 H) |
| 1.023 | 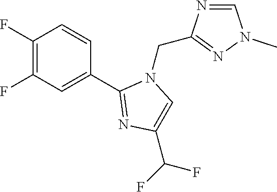 [SMILES: Cn1cnc(Cn2cc(C(F)F)nc2-c2ccc(F)c(F)c2)n1] | ¹H NMR (400 MHz, CHLORO- FORM-d) δ = 8.04 (s, 1 H), 7.76 (dd, 1 H), 7.73 (dd, 1 H), 7.41 (t, 1 H), 7.24-7.32 (m, 1 H), 6.67 (t, 1 H), 5.18 (s, 2 H), 3.94 (s, 3 H) |
| 1.024 | 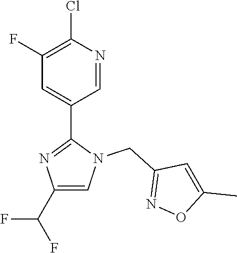 [SMILES: Cc1cc(Cn2cc(C(F)F)nc2-c2cnc(Cl)c(F)c2)no1] | ¹H NMR (400 MHz, CHLORO- FORM-d) δ = 8.47 (d, 1 H), 7.86 (dd, 1 H), 7.37 (t, 1 H), 6.68 (t, 1 H), 5.89 (d, 1 H), 5.22 (s, 2 H), 2.44 (d, 3 H) |
| 1.025 | 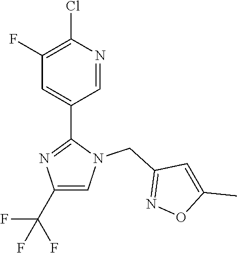 [SMILES: Cc1cc(Cn2cc(C(F)(F)F)nc2-c2cnc(Cl)c(F)c2)no1] | ¹H NMR (400 MHz, chloroform-d) δ = 8.50 (d, 1H), 7.90 (dd, 1H), 7.48 (q, 1H), 5.91 (d, 1H), 5.25 (s, 2H), 2.47 (d, 3H) |
| 1.026 | 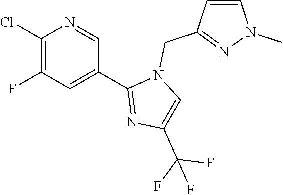 [SMILES: Cn1ccc(Cn2cc(C(F)(F)F)nc2-c2cnc(Cl)c(F)c2)n1] | ¹H NMR (400 MHz, chloroform-d) δ = 8.63 (d, 1H), 8.00 (dd, 1H), 7.46 (d, 1H), 7.36 (d, 1H), 6.14 (d, 1H), 5.16 (s, 2H), 3.91 (s, 3H) |
| 1.027 | 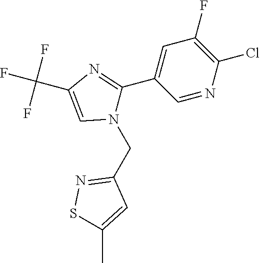 [SMILES: Cc1cc(Cn2cc(C(F)(F)F)nc2-c2cnc(Cl)c(F)c2)ns1] | ¹H NMR (400 MHz, chloroform-d) δ = 8.51 (d, 1H), 7.95 (dd, 1H), 7.49 (d, 1H), 6.82 (d, 1H), 5.27 (s, 2H), 2.60 (d, 3H) |
| 1.028 | 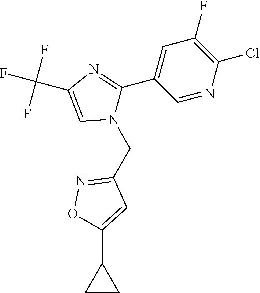 [SMILES: Fc1cc(-c2nc(C(F)(F)F)cn2Cc2cc(C3CC3)on2)cnc1Cl] | ¹H NMR (400 MHz, chloroform-d) δ = 8.44 (d, 1H), 7.85 (dd, 1H), 7.49 (d, 1H), 5.92 (s, 1H), 5.25 (s, 2H), 1.99 (tt, 1H), 1.13-1.05 (m, 2H), 0.89-0.79 (m, 2H) |

TABLE 1b
Compounds of the Present Invention

| | | LCMS | | |
|---|---|---|---|---|
| | | | [M + H] | |
| Compound | STRUCTURE | RT (min) | (measured) | Method |
| 1.029 | 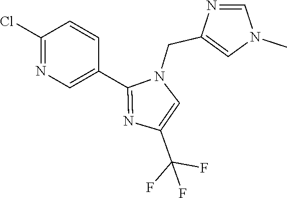 [SMILES: Cn1cnc(Cn2cc(C(F)(F)F)nc2-c2ccc(Cl)nc2)c1] | 0.713 | 342.2 | 2 |
| 1.030 | 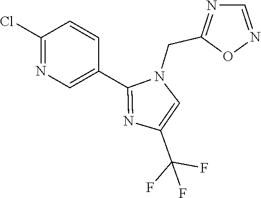 [SMILES: FC(F)(F)c1cn(Cc2ncno2)c(-c2ccc(Cl)nc2)n1] | 0.863 | 330 | 2 |
| 1.031 | 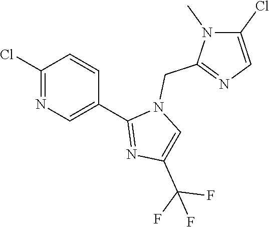 [SMILES: Cn1c(Cl)cnc1Cn1cc(C(F)(F)F)nc1-c1ccc(Cl)nc1] | 1.08 | 376 | 2 |
| 1.032 | 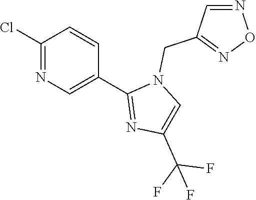 [SMILES: FC(F)(F)c1cn(Cc2cnon2)c(-c2ccc(Cl)nc2)n1] | 1.012 | 330.2 | 2 |
| 1.033 | 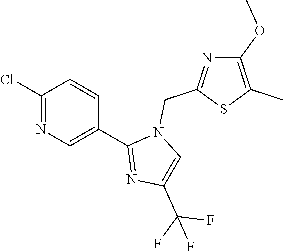 [SMILES: COc1nc(Cn2cc(C(F)(F)F)nc2-c2ccc(Cl)nc2)sc1C] | 1.303 | 389 | 2 |
| 1.034 | 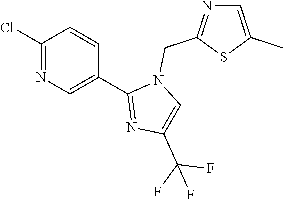 [SMILES: Cc1cnc(Cn2cc(C(F)(F)F)nc2-c2ccc(Cl)nc2)s1] | 1.272 | 359 | 2 |
| 1.035 | 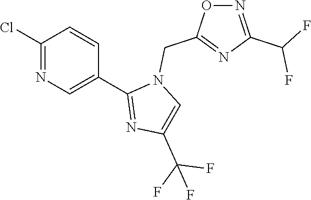 [SMILES: FC(F)c1noc(Cn2cc(C(F)(F)F)nc2-c2ccc(Cl)nc2)n1] | 1.219 | 380 | 2 |
| 1.036 | 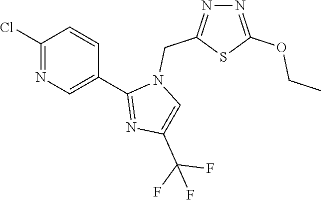 [SMILES: CCOc1nnc(Cn2cc(C(F)(F)F)nc2-c2ccc(Cl)nc2)s1] | 1.324 | 389.9 | 2 |
| 1.037 | 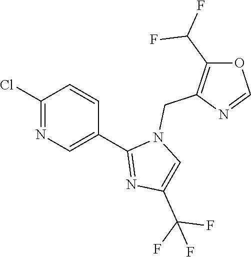 [SMILES: FC(F)c1ocnc1Cn1cc(C(F)(F)F)nc1-c1ccc(Cl)nc1] | 1.296 | 378.9 | 2 |
| 1.038 | 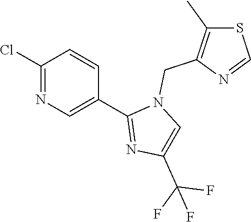 [SMILES: Cc1scnc1Cn1cc(C(F)(F)F)nc1-c1ccc(Cl)nc1] | 1.303 | 359 | 2 |
| 1.039 | 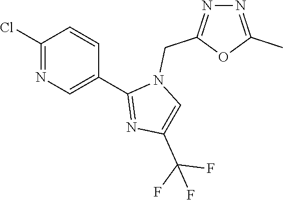 [SMILES: Cc1nnc(Cn2cc(C(F)(F)F)nc2-c2ccc(Cl)nc2)o1] | 0.956 | 344 | 2 |
| 1.040 | 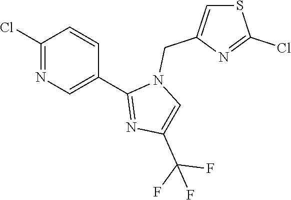 [SMILES: FC(F)(F)c1cn(Cc2csc(Cl)n2)c(-c2ccc(Cl)nc2)n1] | 1.241 | 379 | 2 |
| 1.041 | 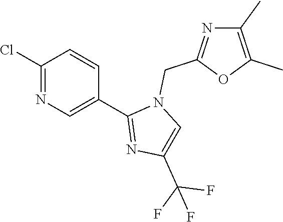 [SMILES: Cc1nc(Cn2cc(C(F)(F)F)nc2-c2ccc(Cl)nc2)oc1C] | 1.176 | 357.2 | 2 |
| 1.042 | 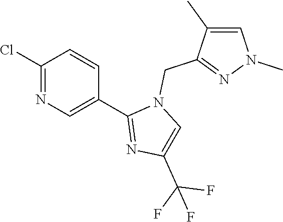 [SMILES: Cc1cn(C)nc1Cn1cc(C(F)(F)F)nc1-c1ccc(Cl)nc1] | 1.125 | 356.2 | 2 |
| 1.043 | 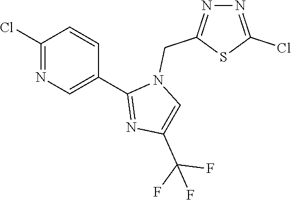 [SMILES: FC(F)(F)c1cn(Cc2nnc(Cl)s2)c(-c2ccc(Cl)nc2)n1] | 1.12 | 380 | 2 |
| 1.044 | 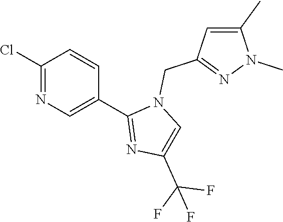 [SMILES: Cc1cc(Cn2cc(C(F)(F)F)nc2-c2ccc(Cl)nc2)nn1C] | 1.119 | 356 | 2 |
| 1.045 | 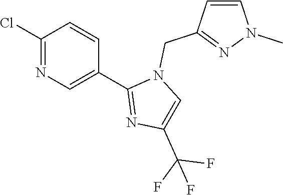 [SMILES: Cn1ccc(Cn2cc(C(F)(F)F)nc2-c2ccc(Cl)nc2)n1] | 2.995 | 342.2 | 3 |
| 1.046 | 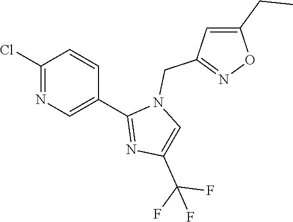 [SMILES: CCc1cc(Cn2cc(C(F)(F)F)nc2-c2ccc(Cl)nc2)no1] | 3.466 | 357.2 | 3 |
| 1.047 | 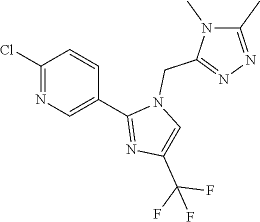 [SMILES: Cc1nnc(Cn2cc(C(F)(F)F)nc2-c2ccc(Cl)nc2)n1C] | 2.21 | 357.2 | 3 |
| 1.048 | 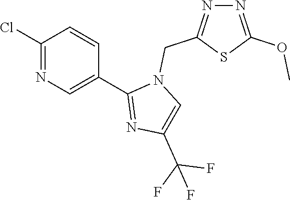 [SMILES: COc1nnc(Cn2cc(C(F)(F)F)nc2-c2ccc(Cl)nc2)s1] | 1.076 | 376 | 2 |
| 1.049 | 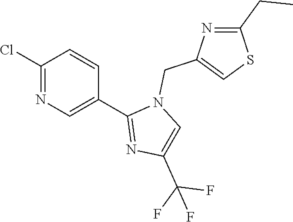 [SMILES: CCc1nc(Cn2cc(C(F)(F)F)nc2-c2ccc(Cl)nc2)cs1] | 1.258 | 373 | 2 |
| 1.050 | 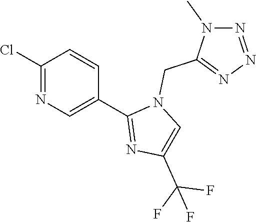 [SMILES: Cn1nnnc1Cn1cc(C(F)(F)F)nc1-c1ccc(Cl)nc1] | 1.113 | 344 | 2 |
| 1.051 | 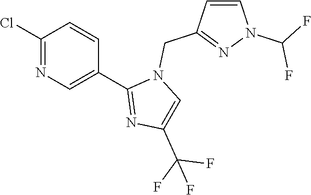 [SMILES: FC(F)n1ccc(Cn2cc(C(F)(F)F)nc2-c2ccc(Cl)nc2)n1] | 1.283 | 378 | 2 |
| 1.052 | 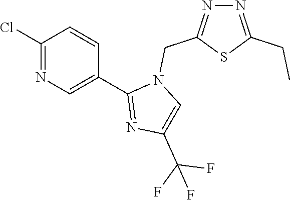 [SMILES: CCc1nnc(Cn2cc(C(F)(F)F)nc2-c2ccc(Cl)nc2)s1] | 1.319 | 374 | 2 |
| 1.053 | 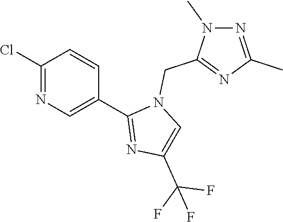 [SMILES: Cc1nc(Cn2cc(C(F)(F)F)nc2-c2ccc(Cl)nc2)n(C)n1] | 1.168 | 357 | 2 |
| 1.054 | 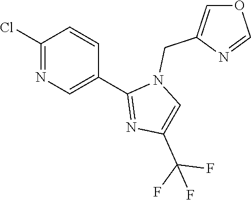 [SMILES: FC(F)(F)c1cn(Cc2cocn2)c(-c2ccc(Cl)nc2)n1] | 1.203 | 329 | 2 |
| 1.055 | 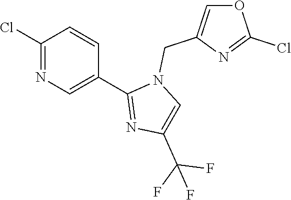 [SMILES: FC(F)(F)c1cn(Cc2coc(Cl)n2)c(-c2ccc(Cl)nc2)n1] | 1.357 | 363 | 2 |
| 1.056 | 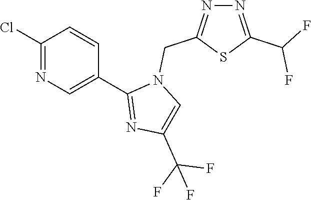 [SMILES: FC(F)c1nnc(Cn2cc(C(F)(F)F)nc2-c2ccc(Cl)nc2)s1] | 1.357 | 396 | 2 |
| 1.057 | 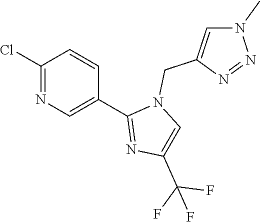 [SMILES: Cn1cc(Cn2cc(C(F)(F)F)nc2-c2ccc(Cl)nc2)nn1] | 1.123 | 343 | 2 |
| 1.058 | 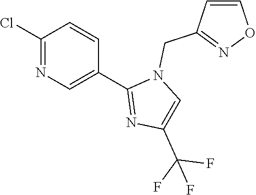 [SMILES: FC(F)(F)c1cn(Cc2ccon2)c(-c2ccc(Cl)nc2)n1] | 1.236 | 329 | 2 |
| 1.059 | 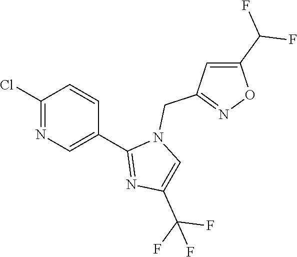 [SMILES: FC(F)c1cc(Cn2cc(C(F)(F)F)nc2-c2ccc(Cl)nc2)no1] | 1.352 | 379 | 2 |
| 1.060 | 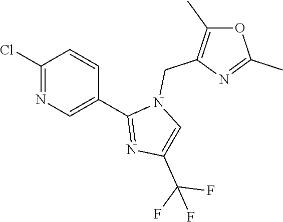 [SMILES: Cc1nc(Cn2cc(C(F)(F)F)nc2-c2ccc(Cl)nc2)c(C)o1] | 1.315 | 357 | 2 |
| 1.061 | 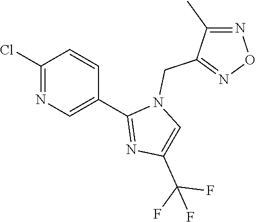 [SMILES: Cc1nonc1Cn1cc(C(F)(F)F)nc1-c1ccc(Cl)nc1] | 1.317 | 344 | 2 |
| 1.062 | 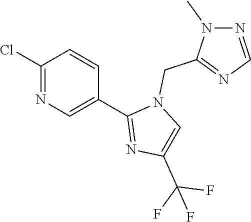 [SMILES: Cn1ncnc1Cn1cc(C(F)(F)F)nc1-c1ccc(Cl)nc1] | 1.08 | 343 | 2 |
| 1.063 | 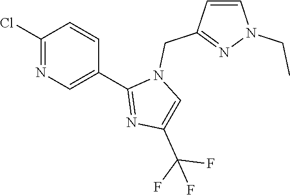 [SMILES: CCn1ccc(Cn2cc(C(F)(F)F)nc2-c2ccc(Cl)nc2)n1] | 1.151 | 356.2 | 2 |
| 1.064 | 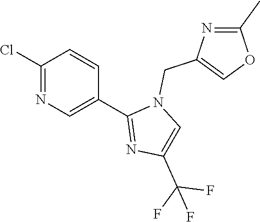 [SMILES: Cc1nc(Cn2cc(C(F)(F)F)nc2-c2ccc(Cl)nc2)co1] | 1.264 | 343 | 2 |
| 1.065 | 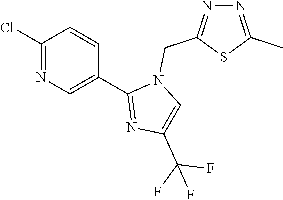 [SMILES: Cc1nnc(Cn2cc(C(F)(F)F)nc2-c2ccc(Cl)nc2)s1] | 1.18 | 360 | 2 |
| 1.066 | 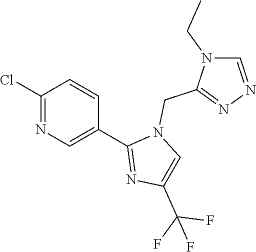 [SMILES: CCn1cnnc1Cn1cc(C(F)(F)F)nc1-c1ccc(Cl)nc1] | 1.035 | 357 | 2 |
| 1.067 | 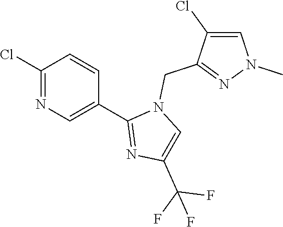 [SMILES: Cn1cc(Cl)c(Cn2cc(C(F)(F)F)nc2-c2ccc(Cl)nc2)n1] | 1.398 | 376 | 2 |
| 1.068 | 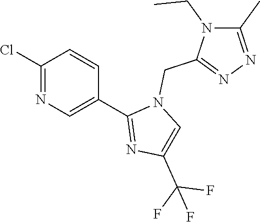 [SMILES: CCn1c(C)nnc1Cn1cc(C(F)(F)F)nc1-c1ccc(Cl)nc1] | 1.045 | 371.2 | 2 |
| 1.069 | 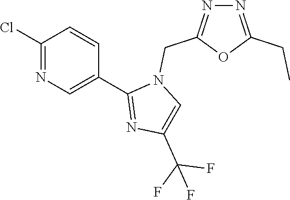 [SMILES: CCc1nnc(Cn2cc(C(F)(F)F)nc2-c2ccc(Cl)nc2)o1] | 1.215 | 358 | 2 |
| 1.070 | 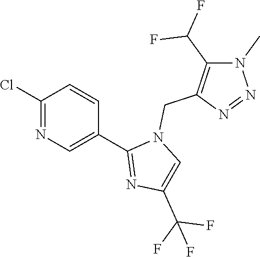 [SMILES: Cn1nnc(Cn2cc(C(F)(F)F)nc2-c2ccc(Cl)nc2)c1C(F)F] | 1.26 | 393 | 2 |
| 1.071 | 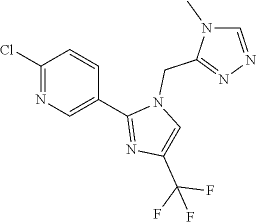 [SMILES: Cn1cnnc1Cn1cc(C(F)(F)F)nc1-c1ccc(Cl)nc1] | 0.971 | 343 | 2 |
| 1.072 | 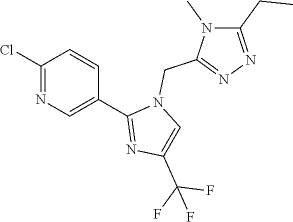 [SMILES: CCc1nnc(Cn2cc(C(F)(F)F)nc2-c2ccc(Cl)nc2)n1C] | 1.036 | 371 | 2 |
| 1.073 | 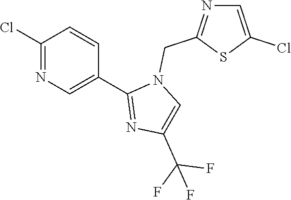 [SMILES: FC(F)(F)c1cn(Cc2ncc(Cl)s2)c(-c2ccc(Cl)nc2)n1] | 1.422 | 379 | 2 |
| 1.074 | 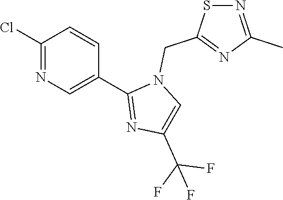 [SMILES: Cc1nsc(Cn2cc(C(F)(F)F)nc2-c2ccc(Cl)nc2)n1] | 1.286 | 360 | 2 |
| 1.075 | 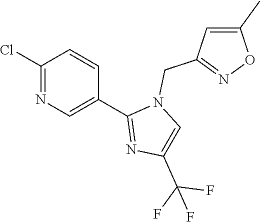 [SMILES: Cc1cc(Cn2cc(C(F)(F)F)nc2-c2ccc(Cl)nc2)no1] | 1.309 | 343 | 2 |
| 1.076 | 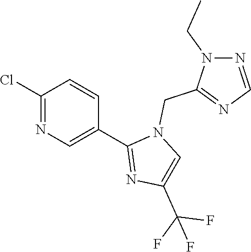 [SMILES: CCn1ncnc1Cn1cc(C(F)(F)F)nc1-c1ccc(Cl)nc1] | 1.158 | 357 | 2 |
| 1.077 | 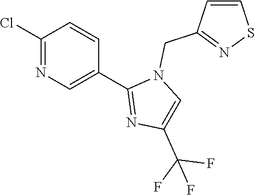 [SMILES: FC(F)(F)c1cn(Cc2ccsn2)c(-c2ccc(Cl)nc2)n1] | 1.301 | 345 | 2 |
| 1.078 | 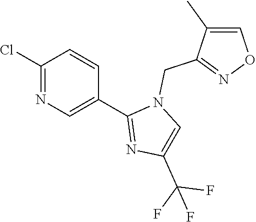 [SMILES: Cc1conc1Cn1cc(C(F)(F)F)nc1-c1ccc(Cl)nc1] | 1.291 | 343 | 2 |
| 1.079 | 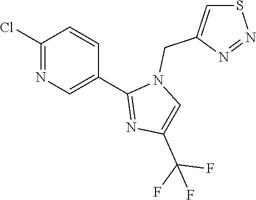 [SMILES: FC(F)(F)c1cn(Cc2csnn2)c(-c2ccc(Cl)nc2)n1] | 1.236 | 346 | 2 |
| 1.080 | 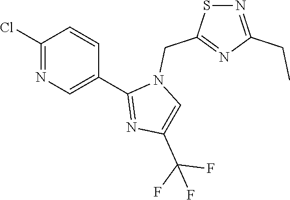 [SMILES: CCc1nsc(Cn2cc(C(F)(F)F)nc2-c2ccc(Cl)nc2)n1] | 1.382 | 374 | 2 |
| 1.081 | 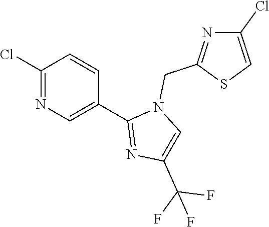 [SMILES: FC(F)(F)c1cn(Cc2nc(Cl)cs2)c(-c2ccc(Cl)nc2)n1] | 1.381 | 379 | 2 |
| 1.082 | 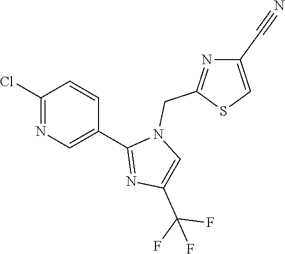 [SMILES: N#Cc1csc(Cn2cc(C(F)(F)F)nc2-c2ccc(Cl)nc2)n1] | 1.331 | 370 | 2 |
| 1.083 | 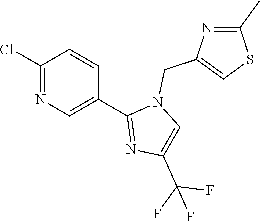 [SMILES: Cc1nc(Cn2cc(C(F)(F)F)nc2-c2ccc(Cl)nc2)cs1] | 1.351 | 359.1 | 2 |
| 1.084 | 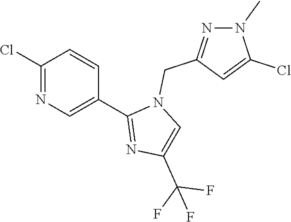 [SMILES: Cn1nc(Cn2cc(C(F)(F)F)nc2-c2ccc(Cl)nc2)cc1Cl] | 1.44 | 376 | 2 |
| 1.085 | 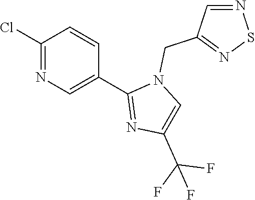 [SMILES: FC(F)(F)c1cn(Cc2cnsn2)c(-c2ccc(Cl)nc2)n1] | 1.296 | 346 | 2 |
| 1.086 | 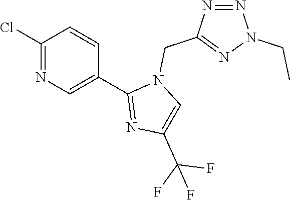 [SMILES: CCn1nnc(Cn2cc(C(F)(F)F)nc2-c2ccc(Cl)nc2)n1] | 1.114 | 358 | 2 |
| 1.087 | 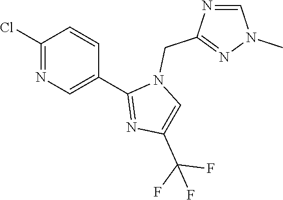 [SMILES: Cn1cnc(Cn2cc(C(F)(F)F)nc2-c2ccc(Cl)nc2)n1] | 1.113 | 343 | 2 |
| 1.088 | 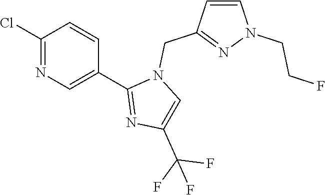 [SMILES: FCCn1ccc(Cn2cc(C(F)(F)F)nc2-c2ccc(Cl)nc2)n1] | 1.273 | 374 | 2 |
| 1.089 | 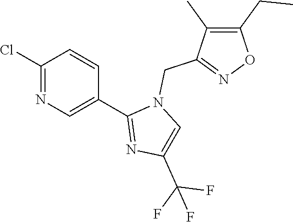 [SMILES: CCc1onc(Cn2cc(C(F)(F)F)nc2-c2ccc(Cl)nc2)c1C] | 1.424 | 371 | 2 |
| 1.090 | 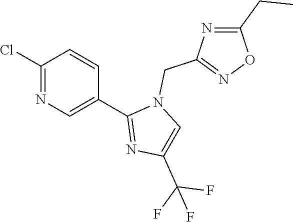 [SMILES: CCc1nc(Cn2cc(C(F)(F)F)nc2-c2ccc(Cl)nc2)no1] | 1.343 | 358 | 2 |
| 1.091 | 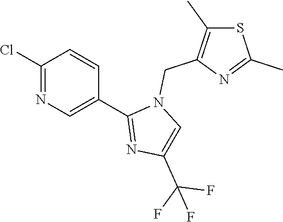 [SMILES: Cc1nc(Cn2cc(C(F)(F)F)nc2-c2ccc(Cl)nc2)c(C)s1] | 1.403 | 373 | 2 |
| 1.092 | 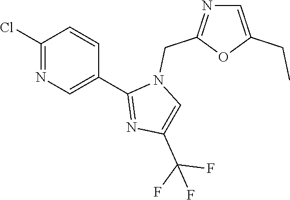 [SMILES: CCc1cnc(Cn2cc(C(F)(F)F)nc2-c2ccc(Cl)nc2)o1] | 1.358 | 357 | 2 |
| 1.093 | 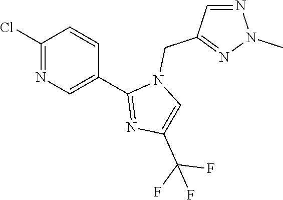 [SMILES: Cn1ncc(Cn2cc(C(F)(F)F)nc2-c2ccc(Cl)nc2)n1] | 1.246 | 343 | 2 |
| 1.094 | 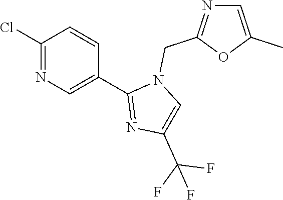 [SMILES: Cc1cnc(Cn2cc(C(F)(F)F)nc2-c2ccc(Cl)nc2)o1] | 1.276 | 343 | 2 |
| 1.095 | 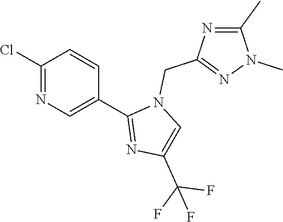 [SMILES: Cc1nc(Cn2cc(C(F)(F)F)nc2-c2ccc(Cl)nc2)nn1C] | 1.199 | 357 | 2 |
| 1.096 | 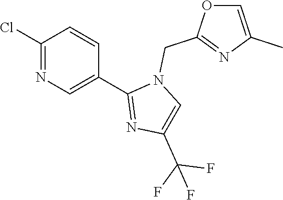 [SMILES: Cc1coc(Cn2cc(C(F)(F)F)nc2-c2ccc(Cl)nc2)n1] | 1.28 | 343 | 2 |
| 1.097 | 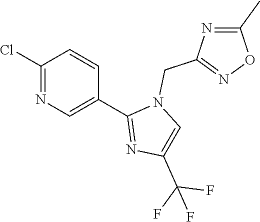 [SMILES: Cc1nc(Cn2cc(C(F)(F)F)nc2-c2ccc(Cl)nc2)no1] | 1.254 | 344 | 2 |
| 1.098 | 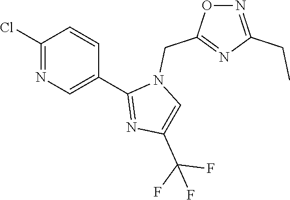 [SMILES: CCc1noc(Cn2cc(C(F)(F)F)nc2-c2ccc(Cl)nc2)n1] | 1.334 | 357.9 | 2 |
| 1.099 | 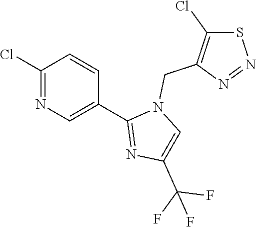 [SMILES: FC(F)(F)c1cn(Cc2nnsc2Cl)c(-c2ccc(Cl)nc2)n1] | 1.349 | 380 | 2 |
| 1.100 | 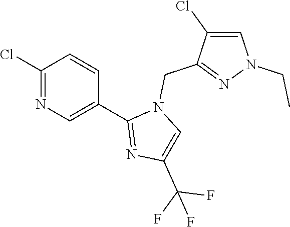 [SMILES: CCn1cc(Cl)c(Cn2cc(C(F)(F)F)nc2-c2ccc(Cl)nc2)n1] | 1.418 | 390 | 2 |
| 1.101 | 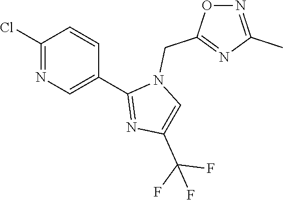 [SMILES: Cc1noc(Cn2cc(C(F)(F)F)nc2-c2ccc(Cl)nc2)n1] | 1.253 | 344 | 2 |
| 1.102 | 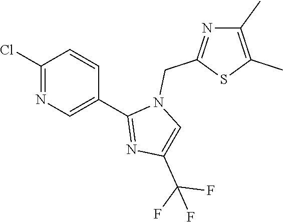 [SMILES: Cc1nc(Cn2cc(C(F)(F)F)nc2-c2ccc(Cl)nc2)sc1C] | 1.417 | 373 | 2 |
| 1.103 | 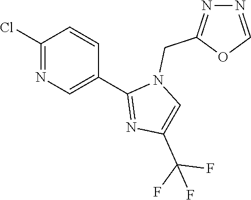 [SMILES: FC(F)(F)c1cn(Cc2nnco2)c(-c2ccc(Cl)nc2)n1] | 1.146 | 330 | 2 |
| 1.104 | 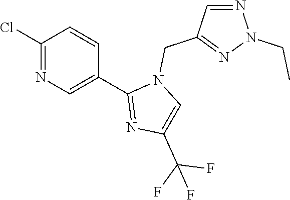 [SMILES: CCn1ncc(Cn2cc(C(F)(F)F)nc2-c2ccc(Cl)nc2)n1] | 1.313 | 357 | 2 |
| 1.105 | 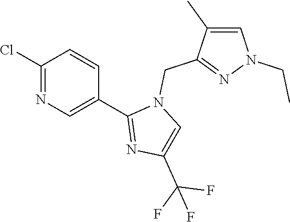 [SMILES: CCn1cc(C)c(Cn2cc(C(F)(F)F)nc2-c2ccc(Cl)nc2)n1] | 1.343 | 371 | 1 |
| 1.106 | 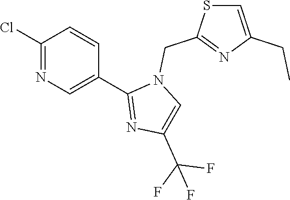 [SMILES: CCc1csc(Cn2cc(C(F)(F)F)nc2-c2ccc(Cl)nc2)n1] | 1.473 | 373 | 2 |
| 1.107 | 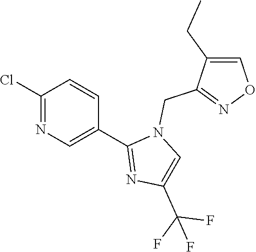 [SMILES: CCc1conc1Cn1cc(C(F)(F)F)nc1-c1ccc(Cl)nc1] | 1.362 | 357 | 2 |
| 1.108 | 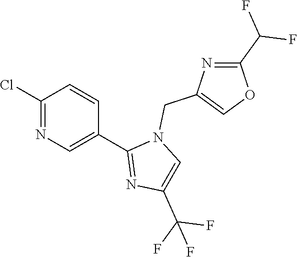 [SMILES: FC(F)c1nc(Cn2cc(C(F)(F)F)nc2-c2ccc(Cl)nc2)co1] | 1.351 | 379 | 2 |
| 1.109 | 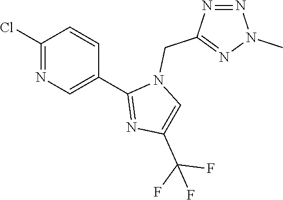 [SMILES: Cn1nnc(Cn2cc(C(F)(F)F)nc2-c2ccc(Cl)nc2)n1] | 1.207 | 344 | 2 |
| 1.110 | 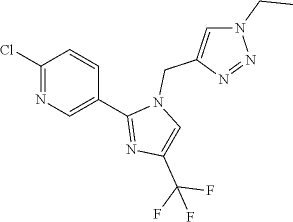 [SMILES: CCn1cc(Cn2cc(C(F)(F)F)nc2-c2ccc(Cl)nc2)nn1] | 1.21 | 357.1 | 2 |
| 1.111 | 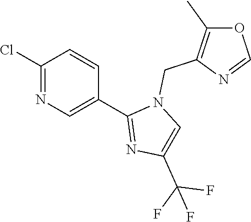 [SMILES: Cc1ocnc1Cn1cc(C(F)(F)F)nc1-c1ccc(Cl)nc1] | 1.312 | 343 | 2 |
| 1.112 | 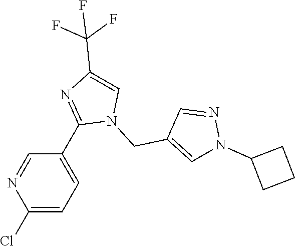 [SMILES: FC(F)(F)c1cn(Cc2cnn(C3CCC3)c2)c(-c2ccc(Cl)nc2)n1] | 1.41 | 382.5 | 1 |
| 1.113 | 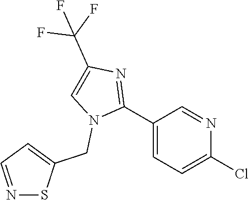 [SMILES: FC(F)(F)c1cn(Cc2ccns2)c(-c2ccc(Cl)nc2)n1] | 1.23 | 345.4 | 1 |
| 1.114 | 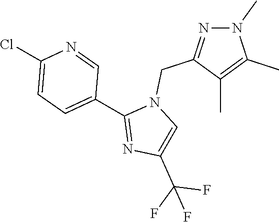 [SMILES: Cc1c(Cn2cc(C(F)(F)F)nc2-c2ccc(Cl)nc2)nn(C)c1C] | 1.38 | 370.5 | 1 |
| 1.115 | 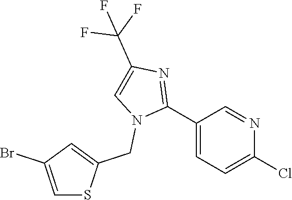 [SMILES: FC(F)(F)c1cn(Cc2cc(Br)cs2)c(-c2ccc(Cl)nc2)n1] | 1.63 | 422.3 | 1 |
| 1.116 | 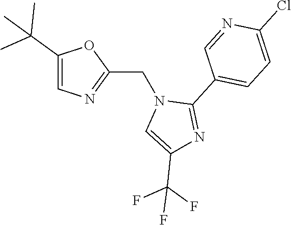 [SMILES: CC(C)(C)c1cnc(Cn2cc(C(F)(F)F)nc2-c2ccc(Cl)nc2)o1] | 1.62 | 385.5 | 1 |
| 1.117 | 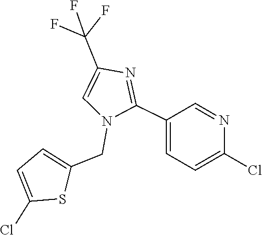 [SMILES: FC(F)(F)c1cn(Cc2ccc(Cl)s2)c(-c2ccc(Cl)nc2)n1] | 1.66 | 378.3 | 1 |
| 1.118 | 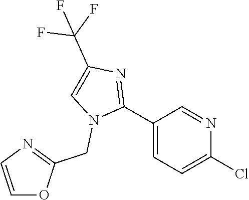 [SMILES: FC(F)(F)c1cn(Cc2ncco2)c(-c2ccc(Cl)nc2)n1] | 1.13 | 329.4 | 1 |
| 1.119 | 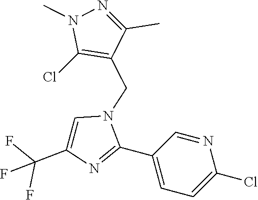 [SMILES: Cc1nn(C)c(Cl)c1Cn1cc(C(F)(F)F)nc1-c1ccc(Cl)nc1] | 1.34 | 390.4 | 1 |
| 1.120 | 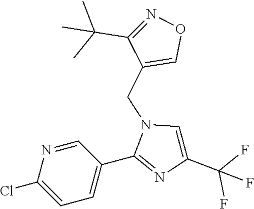 [SMILES: CC(C)(C)c1nocc1Cn1cc(C(F)(F)F)nc1-c1ccc(Cl)nc1] | 1.56 | 385.5 | 1 |
| 1.121 | 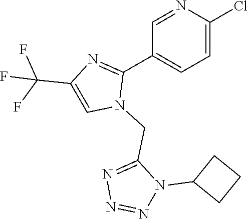 [SMILES: FC(F)(F)c1cn(Cc2nnnn2C2CCC2)c(-c2ccc(Cl)nc2)n1] | 1.3 | 384.4 | 1 |
| 1.122 | 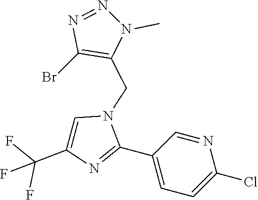 [SMILES: Cn1nnc(Br)c1Cn1cc(C(F)(F)F)nc1-c1ccc(Cl)nc1] | 1.17 | 421.3 | 1 |
| 1.123 | 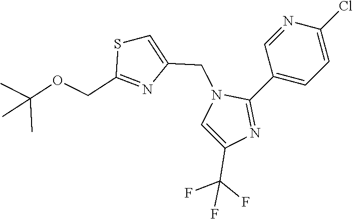 [SMILES: CC(C)(C)OCc1nc(Cn2cc(C(F)(F)F)nc2-c2ccc(Cl)nc2)cs1] | 1.73 | 431.5 | 1 |
| 1.124 | 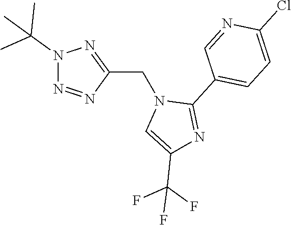 [SMILES: CC(C)(C)n1nnc(Cn2cc(C(F)(F)F)nc2-c2ccc(Cl)nc2)n1] | 1.51 | 386.5 | 1 |
| 1.125 | 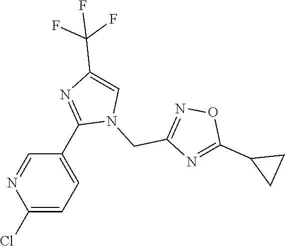 [SMILES: FC(F)(F)c1cn(Cc2noc(C3CC3)n2)c(-c2ccc(Cl)nc2)n1] | 1.41 | 370.4 | 1 |
| 1.126 | 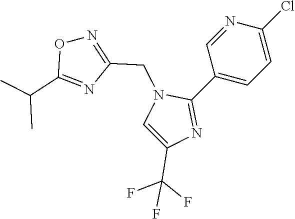 [SMILES: CC(C)c1nc(Cn2cc(C(F)(F)F)nc2-c2ccc(Cl)nc2)no1] | 1.5 | 372.4 | 1 |
| 1.127 | 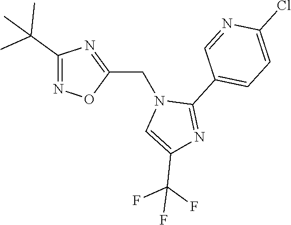 [SMILES: CC(C)(C)c1noc(Cn2cc(C(F)(F)F)nc2-c2ccc(Cl)nc2)n1] | 1.61 | 386.5 | 1 |
| 1.128 | 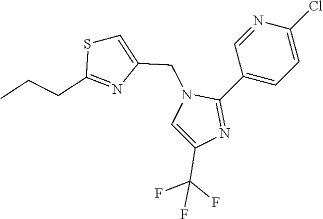 [SMILES: CCCc1nc(Cn2cc(C(F)(F)F)nc2-c2ccc(Cl)nc2)cs1] | 1.64 | 387.4 | 1 |
| 1.129 | 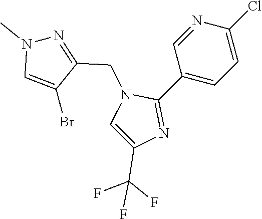 [SMILES: Cn1cc(Br)c(Cn2cc(C(F)(F)F)nc2-c2ccc(Cl)nc2)n1] | 1.39 | 420.3 | 1 |
| 1.130 | 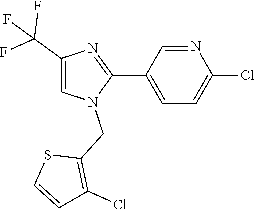 [SMILES: FC(F)(F)c1cn(Cc2sccc2Cl)c(-c2ccc(Cl)nc2)n1] | 1.57 | 378.3 | 1 |
| 1.131 | 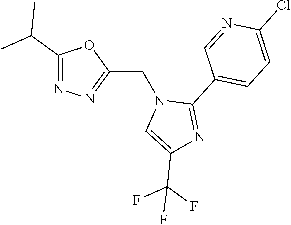 [SMILES: CC(C)c1nnc(Cn2cc(C(F)(F)F)nc2-c2ccc(Cl)nc2)o1] | 1.29 | 372.4 | 1 |
| 1.132 | 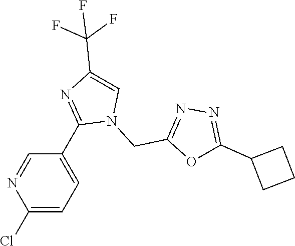 [SMILES: FC(F)(F)c1cn(Cc2nnc(C3CCC3)o2)c(-c2ccc(Cl)nc2)n1] | 1.34 | 384.4 | 1 |
| 1.133 | 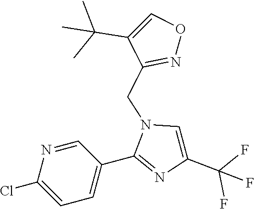 [SMILES: CC(C)(C)c1conc1Cn1cc(C(F)(F)F)nc1-c1ccc(Cl)nc1] | 1.59 | 385.5 | 1 |
| 1.134 | 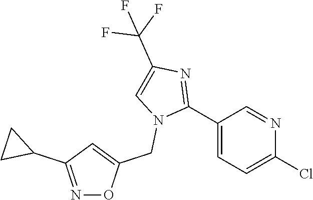 [SMILES: FC(F)(F)c1cn(Cc2cc(C3CC3)no2)c(-c2ccc(Cl)nc2)n1] | 1.4 | 369.4 | 1 |
| 1.135 | 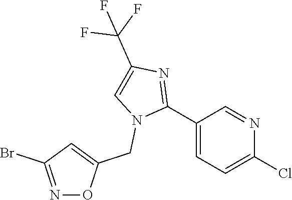 [SMILES: FC(F)(F)c1cn(Cc2cc(Br)no2)c(-c2ccc(Cl)nc2)n1] | 1.41 | 407.3 | 1 |
| 1.136 | 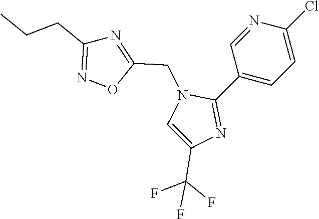 [SMILES: CCCc1noc(Cn2cc(C(F)(F)F)nc2-c2ccc(Cl)nc2)n1] | 1.49 | 372.4 | 1 |
| 1.137 | 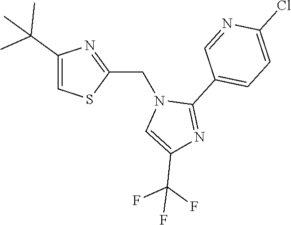 [SMILES: CC(C)(C)c1csc(Cn2cc(C(F)(F)F)nc2-c2ccc(Cl)nc2)n1] | 1.78 | 401.5 | 1 |
| 1.138 | 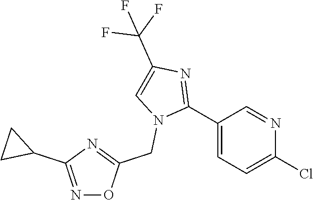 [SMILES: FC(F)(F)c1cn(Cc2nc(C3CC3)no2)c(-c2ccc(Cl)nc2)n1] | 1.4 | 370.4 | 1 |
| 1.139 | 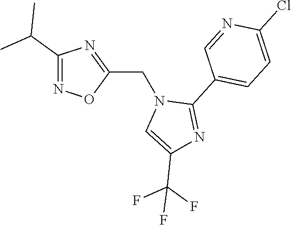 [SMILES: CC(C)c1noc(Cn2cc(C(F)(F)F)nc2-c2ccc(Cl)nc2)n1] | 1.49 | 372.4 | 1 |
| 1.140 | 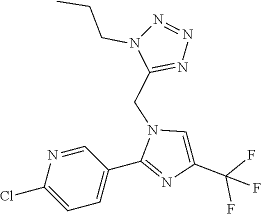 [SMILES: CCCn1nnnc1Cn1cc(C(F)(F)F)nc1-c1ccc(Cl)nc1] | 1.23 | 372.4 | 1 |
| 1.141 | 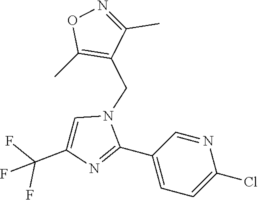 [SMILES: Cc1noc(C)c1Cn1cc(C(F)(F)F)nc1-c1ccc(Cl)nc1] | 1.26 | 357.4 | 1 |
| 1.142 | 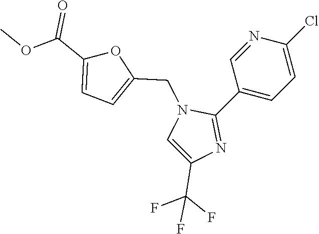 [SMILES: COC(=O)c1ccc(Cn2cc(C(F)(F)F)nc2-c2ccc(Cl)nc2)o1] | 1.35 | 386.4 | 1 |
| 1.143 | 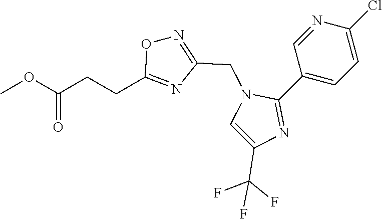 [SMILES: COC(=O)CCc1nc(Cn2cc(C(F)(F)F)nc2-c2ccc(Cl)nc2)no1] | 1.3 | 416.4 | 1 |
| 1.144 | 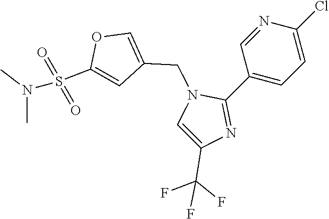 [SMILES: CN(C)S(=O)(=O)c1cc(Cn2cc(C(F)(F)F)nc2-c2ccc(Cl)nc2)co1] | 1.36 | 435.4 | 1 |
| 1.145 | 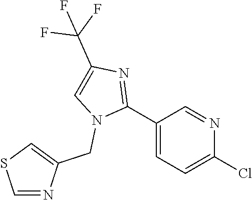 [SMILES: FC(F)(F)c1cn(Cc2cscn2)c(-c2ccc(Cl)nc2)n1] | 1.272 | 345 | 2 |
| 1.146 | 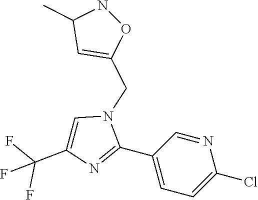 [SMILES: CC1C=C(Cn2cc(C(F)(F)F)nc2-c2ccc(Cl)nc2)ON1] | 1.24 | 343.4 | 1 |
| 1.147 | 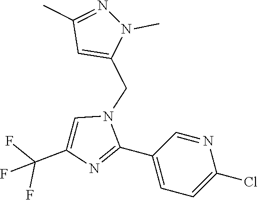 [SMILES: Cc1cc(Cn2cc(C(F)(F)F)nc2-c2ccc(Cl)nc2)n(C)n1] | 1.21 | 356.4 | 1 |
| 1.148 | 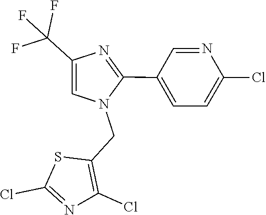 [SMILES: FC(F)(F)c1cn(Cc2sc(Cl)nc2Cl)c(-c2ccc(Cl)nc2)n1] | 1.56 | 413.3 | 1 |
| 1.150 | 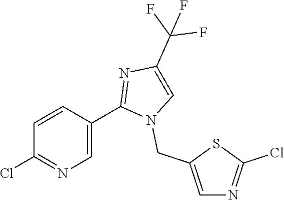 [SMILES: FC(F)(F)c1cn(Cc2cnc(Cl)s2)c(-c2ccc(Cl)nc2)n1] | 1.42 | 379.3 | 1 |

TABLE 1c
Compounds of the Present Invention

| Compound | STRUCTURE | ¹H NMR (500 MHz, CDCl₃) |
|---|---|---|
| 1.151 | 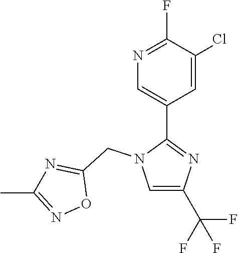 [SMILES: Cc1noc(Cn2cc(C(F)(F)F)nc2-c2cnc(F)c(Cl)c2)n1] | ¹H NMR (400 MHz, chloroform-d) δ = 8.44 (dd, 1H), 8.26 (dd, 1H), 7.56 (d, 1H), 5.37 (s, 2H), 2.45 (s, 3H) |
| 1.152 | 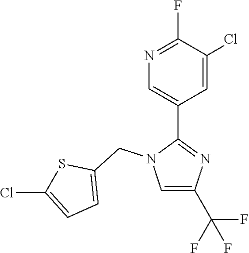 [SMILES: Fc1ncc(-c2nc(C(F)(F)F)cn2Cc2ccc(Cl)s2)cc1Cl] | ¹H NMR (400 MHz, chloroform-d) δ = 8.30 (dd, 1H), 8.14 (dd, 1H), 7.42 (d, 1H), 6.84 (d, 1H), 6.72 (d, 1H), 5.26 (s, 2H) |
| 1.153 | 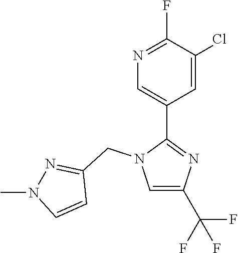 [SMILES: Cn1ccc(Cn2cc(C(F)(F)F)nc2-c2cnc(F)c(Cl)c2)n1] | ¹H NMR (400 MHz, chloroform-d) δ = 8.49- 8.47 (m, 1H), 8.30 (dd, 1H), 7.46-7.44 (m, 1H), 7.36 (d, 1H), 6.13 (d, 1H), 5.14 (s, 2H), 3.90 (s, 3H) |
| 1.154 | 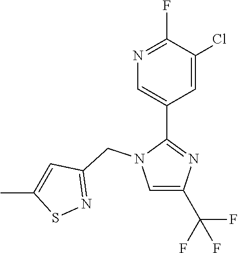 [SMILES: Cc1cc(Cn2cc(C(F)(F)F)nc2-c2cnc(F)c(Cl)c2)ns1] | ¹H NMR (400 MHz, chloroform-d) δ = 8.38- 8.35 (m, 1H), 8.23 (dd, 1H), 7.48-7.46 (m, 1H), 6.80-6.78 (m, 1H), 5.24 (s, 2H), 2.59 (d, 3H). |
| 1.155 | 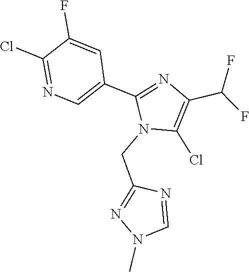 [SMILES: Cn1cnc(Cn2c(-c3cnc(Cl)c(F)c3)nc(C(F)F)c2Cl)n1] | ¹H NMR (400 MHz, METHANOL-d4) δ = 8.59 (d, 1 H), 8.37 (s, 1 H), 8.21 (d, 1 H), 6.82 (t, 1 H), 5.38 (s, 2 H), 3.90 (s, 3 H) |
| 1.156 | 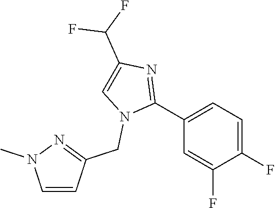 [SMILES: Cn1ccc(Cn2cc(C(F)F)nc2-c2ccc(F)c(F)c2)n1] | ¹H NMR (400 MHz, ACETONITRILE-d3) δ = 7.68 (m, 1 H), 7.65 (m, 1 H), 7.44 (m, 2 H), 7.39 (m, 1 H), 6.71.(t, 1H), 6.13 (d , 1 H), 5.15 (s, 2 H), 3.80 (s, 3 H) |
| 1.157 | 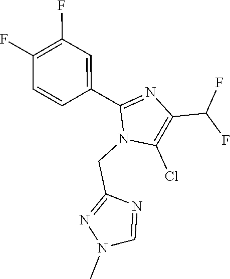 [SMILES: Cn1cnc(Cn2c(-c3ccc(F)c(F)c3)nc(C(F)F)c2Cl)n1] | ¹H NMR (400 MHz, CHLOROFORM-d) δ = 8.04 (s, 1 H), 7.74 (m, 1 H), 7.53-7.59 (m, 1 H), 7.20-7.28 (m, 1 H), 6.70 (t, 1 H), 5.22 (s, 2 H), 3.93 (s, 3H) |
| 1.158 | 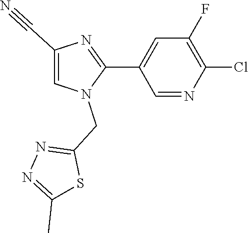 [SMILES: Cc1nnc(Cn2cc(C#N)nc2-c2cnc(Cl)c(F)c2)s1] | ¹H NMR (400 MHz, CHLOROFORM-d) δ = 8.53 (d, 1 H), 7.94 (dd, 1 H), 7.75 (s, 1 H), 5.61 (s, 2 H), 2.84 (s, 3 H) |
| 1.159 | 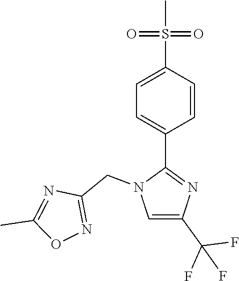 [SMILES: Cc1nc(Cn2cc(C(F)(F)F)nc2-c2ccc(S(C)(=O)=O)cc2)no1] | ¹H NMR (400 MHz, chloroform-d) δ = 8.11- 8.08 (m, 2H), 8.04-8.00 (m, 2H), 7.58-7.56 (m, 1H), 5.25 (s, 2H), 3.10 (s, 3H), 2.65 (s, 3H). |
| 1.160 | 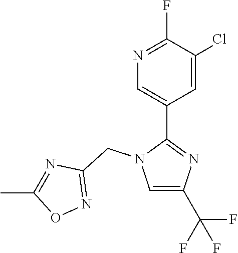 [SMILES: Cc1nc(Cn2cc(C(F)(F)F)nc2-c2cnc(F)c(Cl)c2)no1] | ¹H NMR (400 MHz, chloroform-d) δ = 8.57 (dd, 1H), 8.34 (dd, 1H), 7.54 (d, 1H), 5.23 (s, 2H), 2.65 (s, 3H), |
| 1.161 | 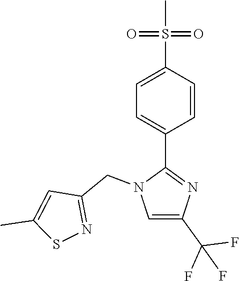 [SMILES: Cc1cc(Cn2cc(C(F)(F)F)nc2-c2ccc(S(C)(=O)=O)cc2)ns1] | ¹H NMR (400 MHz, chloroform-d) δ = 7.99- 7.94 (d, 2H), 7.84-7.79 (d, 2H), 7.40 (d, 1H), 6.69 (s, 1H), 5.20 (s, 2H), 3.01 (s, 3H), 2.51 (s, 3H), |
| 1.162 | 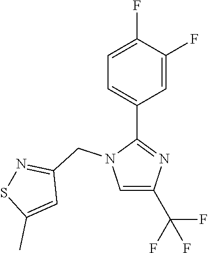 [SMILES: Cc1cc(Cn2cc(C(F)(F)F)nc2-c2ccc(F)c(F)c2)ns1] | ¹H NMR (400 MHz, chloroform-d) δ = 7.52 (m, 1H), 7.43-7.36 (m, 2H), 7.30-7.22 (m, 1H), 6.73 (s, 1H), 5.23 (s, 2H), 2.58 (d, 3H) |
| 1.163 | 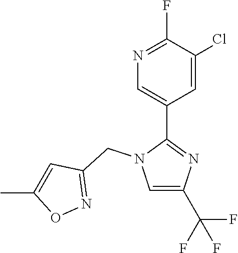 [SMILES: Cc1cc(Cn2cc(C(F)(F)F)nc2-c2cnc(F)c(Cl)c2)no1] | ¹H NMR (400 MHz, chloroform-d) δ = 8.35 (dd, 1H), 8.21 (dd, 1H), 7.46 (d, 1H), 5.89 (s, 1H), 5.22 (s, 2H), 2.46 (s, 3H), |
| 1.164 | 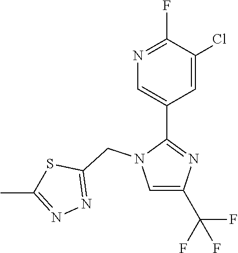 [SMILES: Cc1nnc(Cn2cc(C(F)(F)F)nc2-c2cnc(F)c(Cl)c2)s1] | ¹H NMR (400 MHz, chloroform-d) δ = 8.41 (dd, 1H), 8.26 (dd, 1H), 7.53 (d, 1H), 5.57 (s, 2H), 2.82 (s, 3H) |
| 1.165 | 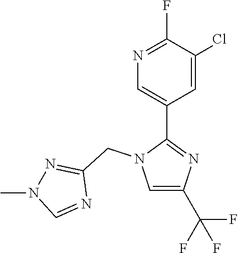 [SMILES: Cn1cnc(Cn2cc(C(F)(F)F)nc2-c2cnc(F)c(Cl)c2)n1] | ¹H NMR (400 MHz, chloroform-d) δ = 8.64 (dd, 1H), 8.44 (dd, 1H), 8.04 (s, 1H), 7.56 (d, 1H), 5.20 (s, 2H), 3.94 (s, 3H) |
| 1.166 | 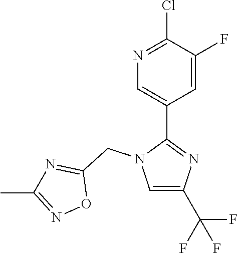 [SMILES: Cc1noc(Cn2cc(C(F)(F)F)nc2-c2cnc(Cl)c(F)c2)n1] | ¹H NMR (400 MHz, chloroform-d) δ = 8.57 (d, 1H), 7.94 (dd, 1H), 7.58-7.54 (m, 1H), 5.39 (s, 2H), 2.45 (s, 3H) |
| 1.167 | 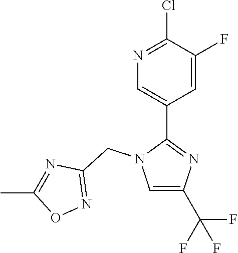 [SMILES: Cc1nc(Cn2cc(C(F)(F)F)nc2-c2cnc(Cl)c(F)c2)no1] | ¹H NMR (400 MHz, chloroform-d) δ = 8.70 (d, 1H), 8.03 (dd, 1H), 7.56-7.54 (m, 1H), 5.24 (s, 2H), 2.65 (s, 3H) |
| 1.168 | 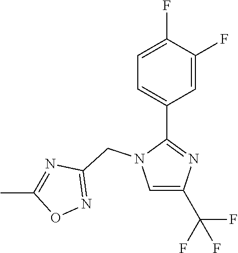 [SMILES: Cc1nc(Cn2cc(C(F)(F)F)nc2-c2ccc(F)c(F)c2)no1] | ¹H NMR (400 MHz, chloroform-d) δ = 7.70- 7.63 (m, 1H), 7.55-7.48 (m, 2H), 7.35-7.27 (m, 1H), 5.21 (s, 2H), 2.65 (s, 3H) |
| 1.169 | 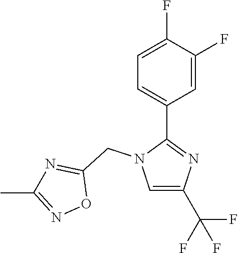 [SMILES: Cc1noc(Cn2cc(C(F)(F)F)nc2-c2ccc(F)c(F)c2)n1] | ¹H NMR (400 MHz, chloroform-d) δ = 7.55 (br d, 1H), 7.51 (d, 1H), 7.43-7.37 (m, 1H), 7.35- 7.26 (m, 1H), 5.36 (s, 2H), 2.45 (s, 3H) |
| 1.170 | 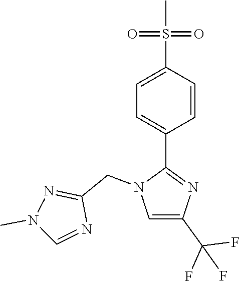 [SMILES: Cn1cnc(Cn2cc(C(F)(F)F)nc2-c2ccc(S(C)(=O)=O)cc2)n1] | ¹H NMR (400 MHz, chloroform-d) δ = 8.14- 8.11 (m, 1H), 8.07 (s, 4H), 7.60 (d, 1H), 5.26 (s, 2H), 3.95 (s, 3H), 3.10 (s, 3H) |
| 1.171 | 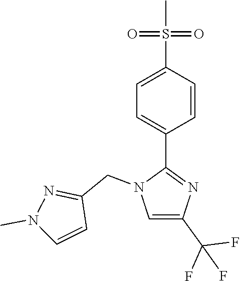 [SMILES: Cn1ccc(Cn2cc(C(F)(F)F)nc2-c2ccc(S(C)(=O)=O)cc2)n1] | ¹H NMR (400 MHz, chloroform-d) δ = 8.07- 8.03 (m, 2H), 8.00-7.95 (m, 2H), 7.48 (d, 1H), 7.37 (d, 1H), 6.12 (d, 1H), 5.19 (s, 2H), 3.90 (s, 3H), 3.09 (s, 3H) |
| 1.172 | 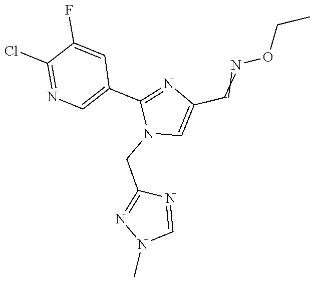 [SMILES: CCON=Cc1cn(Cc2ncn(C)n2)c(-c2cnc(Cl)c(F)c2)n1] | ¹H NMR (400 MHz, METHANOL-d4) δ = 8.62 (d, 1 H), 8.36 (s, 1 H), 8.22 (dd, 1 H), 8.01 (s, 1 H), 7.64 (s, 1 H), 5.34 (s, 2 H), 4.16 (m, 2 H), 3.90 (s, 3 H), 1.28 (t, 3H) |
| 1.173 | 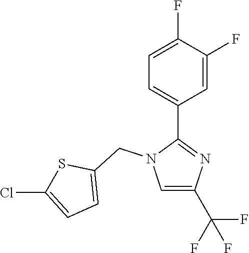 [SMILES: Fc1ccc(-c2nc(C(F)(F)F)cn2Cc2ccc(Cl)s2)cc1F] | ¹H NMR (400 MHz, chloroform-d) δ = 7.46- 7.41 (m, 1H), 7.36 (d, 1H), 7.35-7.24 (m, 2H), 6.81 (d, 1H), 6.72-6.68 (m, 1H), 5.23 (s, 2H) |
| 1.174 | 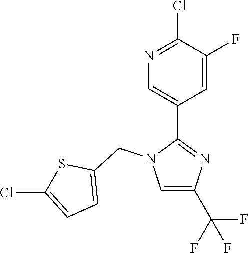 [SMILES: Fc1cc(-c2nc(C(F)(F)F)cn2Cc2ccc(Cl)s2)cnc1Cl] | ¹H NMR (400 MHz, chloroform-d) δ = 8.43 (d, 1H), 7.81 (dd, 1H), 7.44 (d, 1H), 6.83 (d, 1H), 6.77-6.69 (m, 1H), 5.29 (s, 2H) |
| 1.175 | 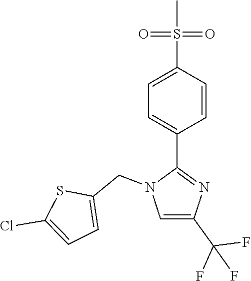 [SMILES: CS(=O)(=O)c1ccc(-c2nc(C(F)(F)F)cn2Cc2ccc(Cl)s2)cc1] | ¹H NMR (400 MHz, chloroform-d) δ = 8.09- 8.05 (m, 2H), 7.85-7.81 (m, 2H), 7.43-7.41 (m, 1H), 6.83 (d, 1H), 6.72-6.70 (m, 1H), 5.27 (s, 2H), 3.10 (s, 3H). |
| 1.176 | 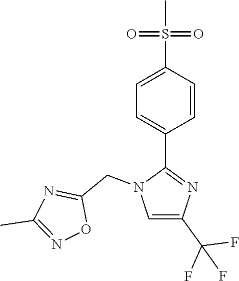 [SMILES: Cc1noc(Cn2cc(C(F)(F)F)nc2-c2ccc(S(C)(=O)=O)cc2)n1] | ¹H NMR (400 MHz, chloroform-d) δ = 8.12- 8.08 (m, 2H), 7.94-7.90 (m, 2H), 7.58-7.56 (m, 1H), 5.38 (s, 2H), 3.10 (s, 3H), 2.46 (s, 3H) |
| 1.177 | 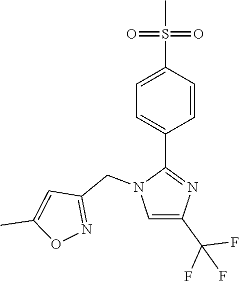 [SMILES: Cc1cc(Cn2cc(C(F)(F)F)nc2-c2ccc(S(C)(=O)=O)cc2)no1] | ¹H NMR (400 MHz, chloroform-d) δ = 8.09- 8.06 (m, 2H), 7.90-7.87 (m, 2H), 7.47-7.45 (m, 1H), 5.86 (d, 1H), 5.24 (s, 2H), 3.09 (s, 3H), 2.46 (d, 3H) |
| 1.178 | 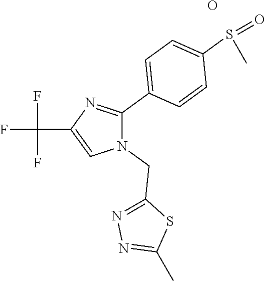 | ¹H NMR (400 MHz, chloroform-d) δ = 8.10 (d, 2H), 7.92 (d, 2H), 7.53 (s, 1H), 5.59 (s, 2H), 3.10 (s, 3H), 2.81 (s, 3H) |
| 1.179 | 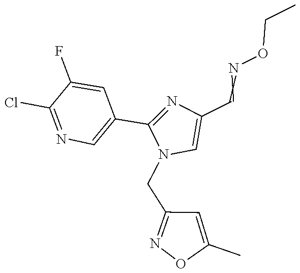 [SMILES: CCON=Cc1cn(Cc2cc(C)on2)c(-c2cnc(Cl)c(F)c2)n1] | Mixture of isomers (3:1) Major isomer ¹H NMR: (400 MHz, CHLOROFORM-d): δ = 8.48 (d, 1H), 8.05 (s, 1H), 7.89 (dd, 1H), 7.39 (s, 1H), 5.86 (s, 1H), 5.20 (s, 2H), 3.97 (s, 3H), 2.44 (d, 3H) Minor isomer ¹H NMR (400 MHz, CHLOROFORM-d): δ = 8.48 (d, 1H), 7.92 (s, 1H), 7.85 (dd, 1H), 7.53 (s, 1H), 5.86 (s, 1H), 5.24 (s, 2H), 4.06 (s, 3H), 2.44 (d, 3H) |
| 1.180 | 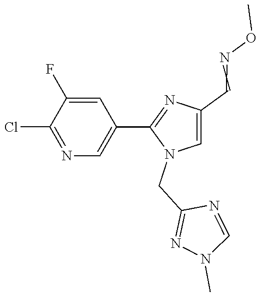 [SMILES: CON=Cc1cn(Cc2ncn(C)n2)c(-c2cnc(Cl)c(F)c2)n1] | Mixture of isomers (3:1) Major isomer: ¹H NMR (400 MHz, METHANOL- d4) δ = 8.60-8.63 (m, 1H), 8.36 (s, 1H), 8.20- 8.25 (m, 1H), 8.01 (s, 1H), 7.64 (s, 1H), 5.34 (s, 2H), 3.90 (s, 6H) Minor isomer: ¹H NMR (400 MHz, METHANOL- d4) δ = 8.60-8.63 (m, 1H), 8.36 (s, 1H), 8.20- 8.25 (m, 1H), 8.09 (s, 1H), 7.39 (s, 1H), 5.37 (m, 2H), 4.02 (s, 3H), 3.90 (s, 3H) |
| 1.181 | 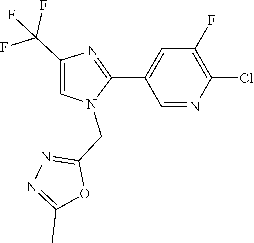 [SMILES: Cc1nnc(Cn2cc(C(F)(F)F)nc2-c2cnc(Cl)c(F)c2)o1] | ¹H NMR (400 MHz, CHLOROFORM-d) δ = 8.59 (d, 1 H), 7.96 (dd, 1 H), 7.58 (s, 1 H), 5.40 (s, 2 H), 2.61 (s, 3 H) |
| 1.182 | 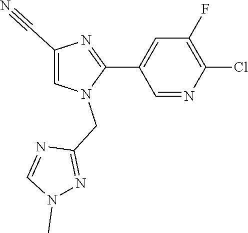 [SMILES: Cn1cnc(Cn2cc(C#N)nc2-c2cnc(Cl)c(F)c2)n1] | ¹H NMR (400 MHz, METHANOL-d4) δ = 8.59 (d, 1 H), 8.36 (s, 1 H), 8.21 (dd, 1 H), 8.14 (s, 1 H), 5.41 (s, 2 H), 3.89 (s, 3 H) |
| 1.183 | 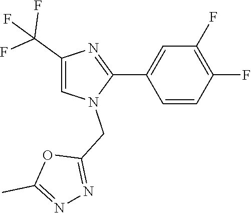 [SMILES: Cc1nnc(Cn2cc(C(F)(F)F)nc2-c2ccc(F)c(F)c2)o1] | ¹H NMR (400 MHz, CHLOROFORM-d) δ = 7.56- 7.62 (m, 1 H), 7.51 (s, 1 H), 7.46 (m, 1 H), 7.30-7.38 (m, 1 H), 5.36 (s, 2 H), 2.60 (s, 3H). |
| 1.184 | 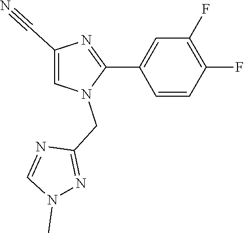 [SMILES: Cn1cnc(Cn2cc(C#N)nc2-c2ccc(F)c(F)c2)n1] | ¹H NMR (400 MHz, CHLOROFORM-d) δ = 8.07 (s, 1 H), 7.72-7.78 (m, 2 H), 7.57-7.61 (m, 1 H), 7.29-7.37 (m, 1 H), 5.23 (s, 2 H), 3.97 (s, 3 H) |
| 1.185 | 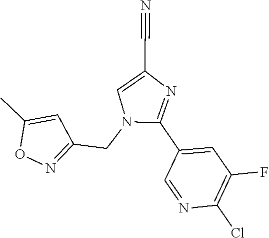 [SMILES: Cc1cc(Cn2cc(C#N)nc2-c2cnc(Cl)c(F)c2)no1] | ¹H NMR (400 MHz, CHLOROFORM-d) δ = 8.47 (d, 1 H), 7.86 (dd, 1 H), 7.67 (s, 1 H), 5.91 (s, 1 H), 5.24 (s, 2 H), 2.47 (s, 3 H) |
| 1.186 | 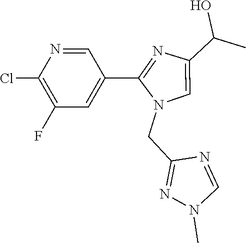 [SMILES: CC(O)c1cn(Cc2ncn(C)n2)c(-c2cnc(Cl)c(F)c2)n1] | ¹H NMR (400 MHz, METHANOL-d4) δ = 8.61 (d, 1 H), 8.36 (s, 1 H), 8.20 (dd, 1 H), 7.23- 7.27 (m, 1 H), 5.28 (s, 2 H), 4.81 (q, 1 H), 3.90 (s, 3 H), 1.49 (d, 3 H) |
| 1.187 | 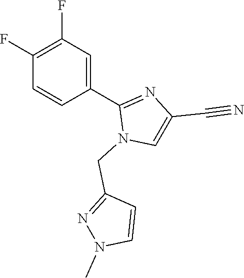 [SMILES: Cn1ccc(Cn2cc(C#N)nc2-c2ccc(F)c(F)c2)n1] | ¹H NMR (400 MHz, CHLOROFORM-d) δ = 7.60 (s, 1 H), 7.59 (m, 1 H), 7.42 (m, 1 H), 7.36 (d, 1 H), 7.28 (m, 1 H), 6.09 (d, 1 H), 5.14 (s, 2 H), 3.91 (s, 3 H), |
| 1.188 | 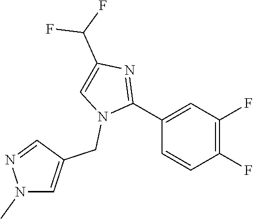 [SMILES: Cn1cc(Cn2cc(C(F)F)nc2-c2ccc(F)c(F)c2)cn1] | ¹H NMR (400 MHz, CHLOROFORM-d) δ = 7.40- 7.45 (m, 1 H), 7.38 (t, 1 H), 7.31 (d, 1 H), 7.25 (d, 1 H), 7.15 (s, 1 H), 7.09 (s, 1 H), 6.69 (t, 1 H), 5.20 (s, 2 H), 3.85 (s, 3 H) |
| 1.189 | 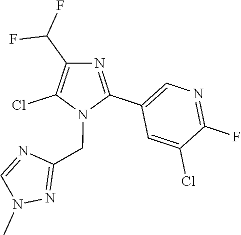 [SMILES: Cn1cnc(Cn2c(-c3cnc(F)c(Cl)c3)nc(C(F)F)c2Cl)n1] | ¹H NMR (400 MHz, chloroform-d) δ = 8.61- .8.58 (m, 1H), 8.42 (dd, 1H), 8.03 (s, 1H), 6.70 (t, 1H), 5.23 (s, 2H), 3.93 (s, 3H) |
| 1.190 | 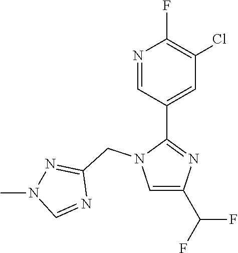 [SMILES: Cn1cnc(Cn2cc(C(F)F)nc2-c2cnc(F)c(Cl)c2)n1] | ¹H NMR (400 MHz, chloroform-d) δ = 8.65- 8.62 (m, 1H), 8.43 (dd, 1H), 8.03 (s, 1H), 7.46 (t, 1H), 6.68 (t, 1H), 5.19 (s, 2H), 3.93 (s, 3H) |
| 1.191 | 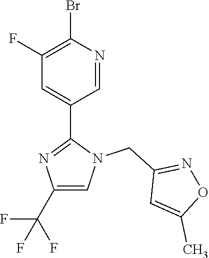 [SMILES: Cc1cc(Cn2cc(C(F)(F)F)nc2-c2cnc(Br)c(F)c2)no1] | ¹H NMR (400 MHz, CHLOROFORM-d) δ = 8.48 (d, 1 H), 7.83 (dd, 1 H), 7.47 (q, 1 H), 5.90 (s, 1 H), 5.23 (s, 2 H), 2.46 (s, 3 H). |
| 1.192 | 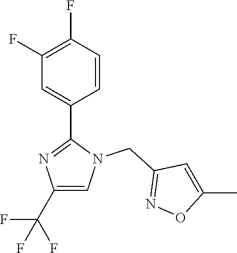 [SMILES: Cc1cc(Cn2cc(C(F)(F)F)nc2-c2ccc(F)c(F)c2)no1] | ¹H NMR (400 MHz, chloroform-d) δ = 7.52 (m, 1H), 7.41-7.35 (m, 2H), 7.33-7.27 (m, 1H), 5.85 (s, 1H), 5.20 (s, 2H), 2.45 (d, 3H), |

### Biological Examples

Seeds of a variety of test species are sown in standard soil in pots Amaranthus palmeri (AMAPA), Amaranthus retoflexus (AMARE), Echinochloa crus-galli (ECHCG), Ipomoea hederacea (IPOHE)). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| | Rate | | | | |
|---|---|---|---|---|---|
| Compound | (g/ha) | AMAPA | AMARE | ECHCG | IPOHE |
| 1.001 | 250 | 4 | 4 | 2 | 3 |
| 1.002 | 250 | 1 | 2 | 1 | 3 |
| 1.003 | 250 | 4 | 4 | 4 | 5 |
| 1.004 | 250 | 1 | 1 | 1 | 2 |
| 1.005 | 250 | 1 | 1 | 1 | 1 |
| 1.006 | 250 | 1 | 1 | 1 | 1 |
| 1.007 | 250 | 5 | 5 | 5 | 5 |
| 1.008 | 250 | 4 | 4 | 2 | 4 |
| 1.009 | 250 | 1 | 2 | 1 | 3 |
| 1.010 | 250 | 1 | 2 | 1 | 3 |
| 1.011 | 250 | 1 | 1 | 1 | 1 |
| 1.012 | 250 | 1 | 1 | 1 | 3 |
| 1.013 | 250 | 5 | 4 | 4 | 4 |
| 1.014 | 250 | 1 | 1 | 1 | 1 |
| 1.015 | 250 | 5 | 5 | 3 | 5 |
| 1.016 | 250 | 1 | 1 | 1 | 1 |
| 1.017 | 250 | 5 | 5 | 2 | 1 |
| 1.018 | 250 | 5 | 5 | 5 | 4 |
| 1.020 | 250 | 5 | 5 | 5 | 4 |
| 1.021 | 250 | 5 | 5 | 4 | 4 |
| 1.024 | 250 | 5 | 5 | 2 | 4 |
| 1.025 | 250 | 4 | 5 | 4 | 2 |
| 1.026 | 250 | 4 | 4 | 3 | 4 |
| 1.027 | 250 | 4 | 4 | 4 | 4 |
| 1.028 | 250 | 3 | 4 | 1 | 1 |
| 1.034 | 250 | 5 | 5 | 5 | 4 |
| 1.044 | 250 | 4 | 4 | 1 | 4 |
| 1.045 | 250 | 5 | 5 | 1 | 4 |
| 1.046 | 250 | 5 | 5 | 4 | 4 |
| 1.051 | 250 | 5 | 5 | 1 | 4 |
| 1.057 | 250 | 4 | 4 | 4 | 4 |
| 1.059 | 250 | 5 | 5 | 5 | 5 |
| 1.063 | 250 | 5 | 5 | 1 | 3 |
| 1.065 | 250 | 5 | 5 | 1 | 1 |
| 1.070 | 250 | 4 | 4 | 1 | 4 |
| 1.075 | 250 | 5 | 5 | 5 | 5 |
| 1.077 | 250 | 4 | 4 | 1 | 1 |
| 1.084 | 250 | 4 | 4 | 1 | 4 |
| 1.086 | 250 | 5 | 5 | 5 | 5 |
| 1.087 | 250 | 5 | 5 | 5 | 5 |
| 1.090 | 250 | 5 | 5 | 4 | 5 |
| 1.092 | 250 | 4 | 4 | 5 | 4 |
| 1.094 | 250 | 5 | 5 | 4 | 3 |
| 1.096 | 250 | 5 | 5 | 4 | 3 |
| 1.097 | 250 | 5 | 5 | 5 | 5 |
| 1.098 | 250 | 4 | 4 | 1 | 3 |
| 1.100 | 250 | 1 | 1 | 1 | 1 |
| 1.101 | 250 | 4 | 4 | 1 | 5 |
| 1.109 | 250 | 5 | 5 | 1 | 5 |
| 1.110 | 250 | 4 | 4 | 1 | 4 |
| 1.113 | 250 | 1 | 1 | 1 | 1 |
| 1.117 | 250 | 5 | 5 | 1 | 2 |
| 1.118 | 250 | 3 | 3 | 1 | 3 |
| 1.125 | 250 | 4 | 5 | 4 | 5 |
| 1.126 | 250 | 1 | 1 | 1 | 1 |
| 1.131 | 250 | 1 | 1 | 1 | 1 |
| 1.134 | 250 | 5 | 5 | 2 | 4 |
| 1.137 | 250 | 1 | 1 | 1 | 1 |
| 1.138 | 250 | 1 | 1 | 1 | 2 |
| 1.139 | 250 | 1 | 1 | 1 | 1 |
| 1.140 | 250 | 1 | 1 | 1 | 1 |
| 1.141 | 250 | 1 | 1 | 1 | 1 |
| 1.144 | 250 | 1 | 1 | 1 | 1 |
| 1.146 | 250 | 5 | 5 | 4 | 4 |
| 1.147 | 250 | 2 | 3 | 1 | 3 |
| 1.148 | 250 | 1 | 1 | 1 | 1 |
| 1.150 | 250 | 1 | 1 | 1 | 1 |
| 1.151 | 250 | 5 | 5 | 3 | 4 |
| 1.152 | 250 | 5 | 5 | 1 | 2 |
| 1.153 | 250 | 5 | 5 | 5 | 4 |
| 1.154 | 250 | 5 | 5 | 5 | 4 |
| 1.159 | 250 | 5 | 5 | 2 | 5 |
| 1.160 | 250 | 5 | 5 | 5 | 4 |
| 1.161 | 250 | 5 | 5 | 5 | 5 |
| 1.162 | 250 | 5 | 5 | 5 | 4 |
| 1.163 | 250 | 5 | 5 | 5 | 4 |
| 1.164 | 250 | 4 | 5 | 2 | 3 |
| 1.165 | 250 | 5 | 5 | 5 | 5 |
| 1.166 | 250 | 4 | 4 | 1 | 2 |
| 1.167 | 250 | 5 | 5 | 5 | 4 |
| 1.168 | 250 | 5 | 5 | 5 | 5 |
| 1.169 | 250 | 5 | 5 | 3 | 3 |
| 1.170 | 250 | 5 | 5 | 2 | 3 |
| 1.171 | 250 | 1 | 1 | 1 | 2 |
| 1.172 | 250 | 2 | 3 | 2 | 2 |
| 1.173 | 250 | 4 | 5 | 2 | 1 |
| 1.174 | 250 | 5 | 5 | 1 | 2 |
| 1.175 | 250 | 4 | 4 | 1 | 2 |
| 1.176 | 250 | 1 | 1 | 1 | 1 |
| 1.177 | 250 | 5 | 5 | 3 | 5 |
| 1.178 | 250 | 1 | 1 | 1 | 1 |
| 1.179 | 250 | 3 | 3 | 1 | 1 |
| 1.180 | 250 | 2 | 1 | 1 | 4 |
| 1.181 | 250 | 4 | 4 | 1 | 1 |
| 1.182 | 250 | 4 | 5 | 3 | 3 |
| 1.183 | 250 | 5 | 5 | 2 | 4 |
| 1.184 | 250 | 5 | 5 | 4 | 5 |
| 1.185 | 250 | 4 | 4 | 1 | 3 |
| 1.192 | 250 | 5 | 5 | 5 | 5 |

TABLE B2
Pre-emergence Test

| | Rate | | | | |
|---|---|---|---|---|---|
| Compound | (g/ha) | AMAPA | AMARE | ECHCG | IPOHE |
| 1.001 | 250 | 5 | 4 | 2 | 2 |
| 1.002 | 250 | 1 | NT | 1 | 1 |
| 1.003 | 250 | 5 | 5 | 5 | 5 |
| 1.004 | 250 | 1 | 1 | 1 | 1 |
| 1.005 | 250 | 5 | 5 | 1 | 1 |
| 1.006 | 250 | 1 | 1 | 1 | 1 |
| 1.007 | 250 | 5 | 5 | 5 | 5 |
| 1.008 | 250 | 5 | 5 | 1 | 3 |
| 1.009 | 250 | 1 | 1 | 1 | 1 |
| 1.010 | 250 | 2 | 5 | 1 | 2 |
| 1.011 | 250 | 1 | 1 | 1 | 1 |
| 1.012 | 250 | NT | 2 | 1 | 1 |
| 1.013 | 250 | 5 | 5 | 5 | 5 |
| 1.014 | 250 | 1 | 1 | 1 | 1 |
| 1.015 | 250 | 5 | 5 | 4 | 5 |
| 1.016 | 250 | 4 | 5 | 1 | 1 |
| 1.017 | 250 | 5 | 5 | 4 | 5 |
| 1.018 | 250 | 5 | 5 | 5 | 5 |
| 1.020 | 250 | 5 | 5 | 5 | 5 |
| 1.021 | 250 | 5 | 5 | 5 | 5 |
| 1.024 | 250 | 5 | 5 | 4 | 5 |
| 1.025 | 250 | 5 | 5 | 4 | 4 |
| 1.026 | 250 | 5 | 5 | 4 | 5 |
| 1.027 | 250 | 5 | 5 | 3 | 4 |
| 1.028 | 250 | 5 | 5 | 1 | 4 |
| 1.034 | 250 | 5 | 5 | 5 | 5 |
| 1.044 | 250 | 5 | 5 | 1 | 1 |
| 1.045 | 250 | 5 | 5 | 1 | 5 |
| 1.046 | 250 | 5 | 5 | 5 | 5 |
| 1.051 | 250 | 5 | 5 | 1 | 5 |
| 1.057 | 250 | 5 | 5 | 4 | 5 |
| 1.059 | 250 | 5 | 5 | 5 | 5 |
| 1.063 | 250 | 5 | 5 | 1 | 5 |
| 1.065 | 250 | 5 | 5 | 1 | 5 |
| 1.070 | 250 | 5 | 5 | 1 | 4 |
| 1.075 | 250 | 5 | 5 | 5 | 5 |
| 1.077 | 250 | 5 | 5 | 1 | 1 |
| 1.084 | 250 | 5 | 5 | 3 | 5 |
| 1.086 | 250 | 5 | 5 | 5 | 5 |
| 1.087 | 250 | 5 | 5 | 5 | 5 |
| 1.090 | 250 | 5 | 5 | 4 | 5 |
| 1.092 | 250 | 5 | 5 | 5 | 5 |
| 1.094 | 250 | 5 | 5 | 5 | 4 |
| 1.096 | 250 | 5 | 5 | 5 | 5 |
| 1.097 | 250 | 5 | 5 | 5 | 5 |
| 1.098 | 250 | 5 | 5 | 1 | 4 |
| 1.100 | 250 | 1 | 1 | 1 | 1 |
| 1.101 | 250 | 5 | 5 | 1 | 5 |
| 1.109 | 250 | 5 | 5 | 5 | 5 |
| 1.110 | 250 | 5 | 5 | 3 | 5 |
| 1.113 | 250 | 1 | 1 | 1 | 1 |
| 1.117 | 250 | 5 | 5 | 1 | 1 |
| 1.118 | 250 | 5 | 5 | 1 | 5 |
| 1.125 | 250 | 5 | 5 | 5 | 5 |
| 1.126 | 250 | 1 | 1 | 1 | 1 |
| 1.131 | 250 | 1 | 5 | 1 | 1 |
| 1.134 | 250 | 5 | 5 | 3 | 4 |
| 1.137 | 250 | 1 | 1 | 1 | 1 |
| 1.138 | 250 | 3 | 3 | 1 | 2 |
| 1.139 | 250 | 1 | 1 | 1 | 1 |
| 1.140 | 250 | 1 | 1 | 1 | 1 |
| 1.141 | 250 | 1 | 1 | 1 | 1 |
| 1.144 | 250 | 1 | 1 | 1 | 1 |
| 1.146 | 250 | 5 | 5 | 5 | 5 |
| 1.147 | 250 | 2 | 2 | 1 | 4 |
| 1.148 | 250 | 1 | 1 | 1 | 1 |
| 1.150 | 250 | 2 | 2 | 1 | 1 |
| 1.151 | 250 | 5 | 5 | 3 | 5 |
| 1.152 | 250 | 5 | 5 | 3 | 1 |
| 1.153 | 250 | 5 | 5 | 5 | 5 |
| 1.154 | 250 | 5 | 5 | 5 | 5 |
| 1.159 | 250 | 5 | 5 | 1 | 5 |
| 1.160 | 250 | 5 | 5 | 5 | 5 |
| 1.161 | 250 | 5 | 5 | 5 | 5 |
| 1.162 | 250 | 5 | 5 | 5 | 3 |
| 1.163 | 250 | 5 | 5 | 5 | 5 |
| 1.164 | 250 | 5 | 5 | 4 | 5 |
| 1.165 | 250 | 5 | 5 | 5 | 5 |
| 1.166 | 250 | 5 | 5 | 1 | 5 |
| 1.167 | 250 | 5 | 5 | 5 | 5 |
| 1.168 | 250 | 5 | 5 | 5 | 5 |
| 1.169 | 250 | 5 | 5 | 4 | 5 |
| 1.170 | 250 | 5 | 5 | 4 | 5 |
| 1.171 | 250 | 4 | 4 | 1 | 3 |
| 1.172 | 250 | 5 | 5 | 5 | 5 |
| 1.173 | 250 | 5 | 5 | 1 | 1 |
| 1.174 | 250 | 5 | 5 | 1 | 1 |
| 1.175 | 250 | 4 | 5 | 2 | 1 |
| 1.176 | 250 | 1 | 1 | 1 | 1 |
| 1.177 | 250 | 5 | 5 | 5 | 5 |
| 1.178 | 250 | 3 | 3 | 1 | 4 |
| 1.179 | 250 | 5 | 4 | 1 | 1 |
| 1.180 | 250 | 4 | 5 | 1 | 5 |
| 1.181 | 250 | 5 | 5 | 3 | 4 |
| 1.182 | 250 | 5 | 5 | 4 | 5 |
| 1.183 | 250 | 5 | 5 | 3 | 5 |
| 1.184 | 250 | 5 | 5 | 5 | 5 |
| 1.185 | 250 | 5 | 5 | 3 | 4 |
| 1.192 | 250 | 5 | 5 | 5 | 5 |

NT = Not tested

1. A compound of Formula (I): 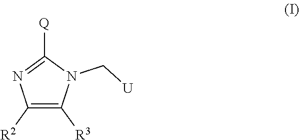
or an agronomically acceptable salt thereof,
wherein
Q is phenyl or a C-linked 6-membered heteroaryl wherein said phenyl or 6-membered heteroaryl is optionally substituted by one or more independent R¹;
U is a 5-membered heteroaryl optionally substituted by one or more independent R⁷;
R¹ is selected from the group consisting of halogen, C₁-C₄ alkyl, C₁-C₄ haloalkyl, C₁-C₄ alkoxy, C₁-C₄ haloalkoxy, C₃-C₆cycloalkyl, C₁-C₄alkoxyC₁-C₃alkyl-, C₁-C₄alkoxyC₁-C₃alkoxy-, C₁-C₄alkoxyC₁-C₃alkoxyC₁-C₃alkyl-, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁴R⁵;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, —S(O)ₚC₁-C₄alkyl, —C(R⁶)═NOR⁸ and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁴R⁵;
R⁴ is selected from the group consisting of hydrogen, C₃-C₄cycloalkyl, C₁-C₄alkyl and C₁-C₄haloalkyl;
R⁵ is selected from the group consisting of hydrogen, C₃-C₄cycloalkyl, C₁-C₄alkyl and C₁-C₄haloalkyl;
R⁶ is hydrogen or C₁-C₂ alkyl;
R⁷ is independently of halogen, C₁-C₄ alkyl, C₃-C₄cycloalkyl, C₁-C₄ haloalkyl, C₁-C₄ alkoxy, C₁-C₄ haloalkoxy, C₁-C₄alkoxyC₁-C₃alkyl-, C₁-C₄alkoxyC₁-C₃alkoxy-, —CN, C₂-C₄alkenyl, C₂-C₄alkynyl, —S(O)ₚC₁-C₄alkyl, —S(O)ₚC₁-C₄haloalkyl, —C(O)OC₁-C₄alkyl, —C(O)NR⁹R¹⁰, —NR¹¹COR¹² and —S(O)ₚNR¹³R¹⁴,
R⁸ is hydrogen or C₁-C₂ alkyl;
R⁹ is selected from the group consisting of hydrogen, C₃-C₄cycloalkyl, C₁-C₄alkyl and C₁-C₄haloalkyl;
R¹⁰ is selected from the group consisting of hydrogen, C₃-C₄cycloalkyl, C₁-C₄alkyl and C₁-C₄haloalkyl;
R¹¹ is selected from the group consisting of hydrogen, C₃-C₄cycloalkyl, C₁-C₄alkyl and C₁-C₄haloalkyl;
R¹² is selected from the group consisting of hydrogen, C₃-C₄cycloalkyl, C₁-C₄alkyl and C₁-C₄haloalkyl;
R¹³ is selected from the group consisting of hydrogen, C₃-C₄cycloalkyl, C₁-C₄alkyl and C₁-C₄haloalkyl;
R¹⁴ is selected from the group consisting of hydrogen, C₃-C₄cycloalkyl, C₁-C₄alkyl and C₁-C₄haloalkyl; and
p=0, 1 or 2.

2. A compound of Formula (I) according to claim 1, wherein U is selected from the group consisting of U1 to U46: 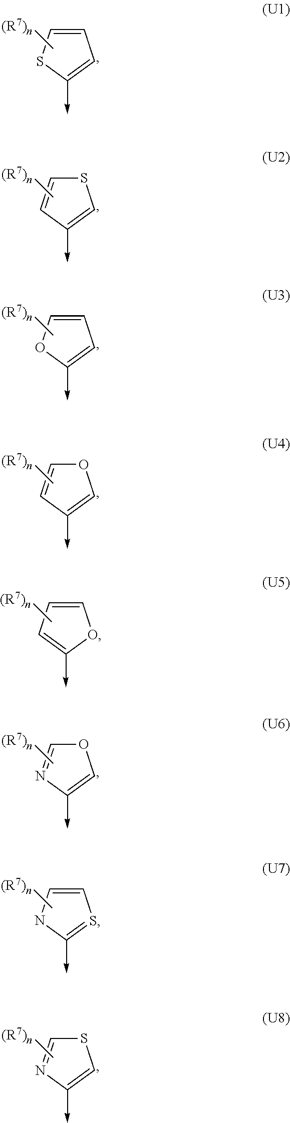 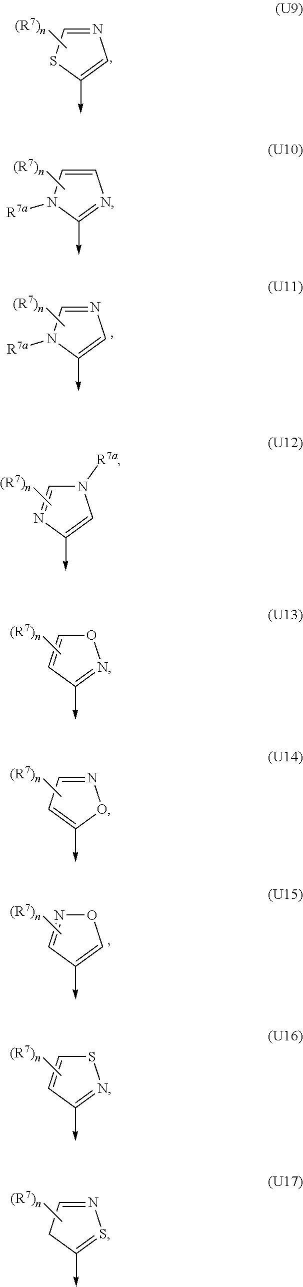 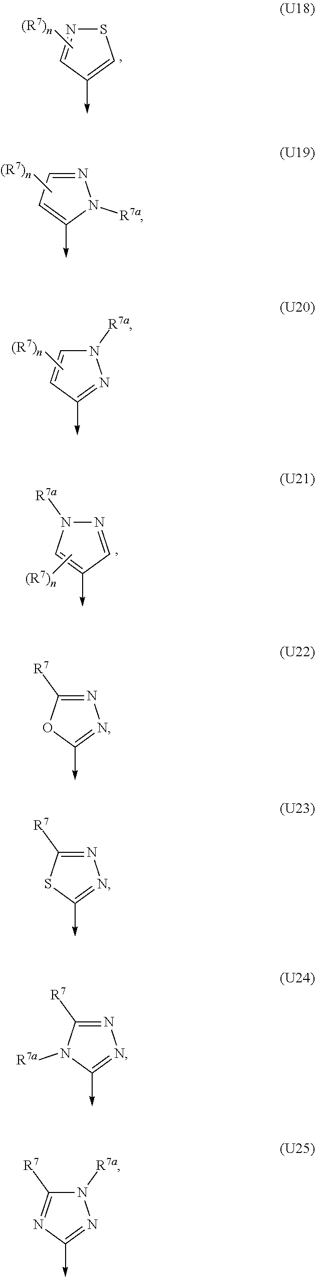 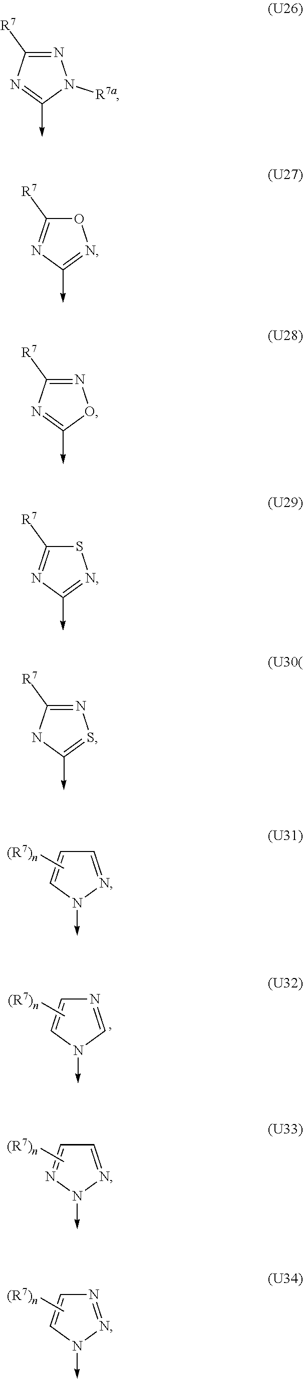 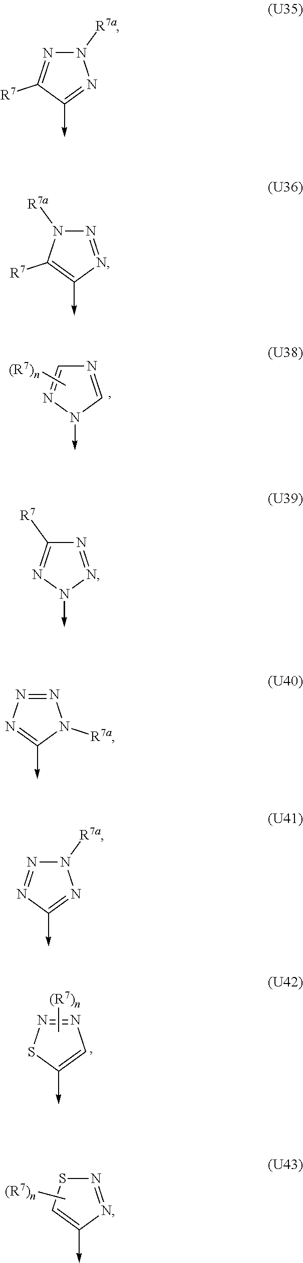 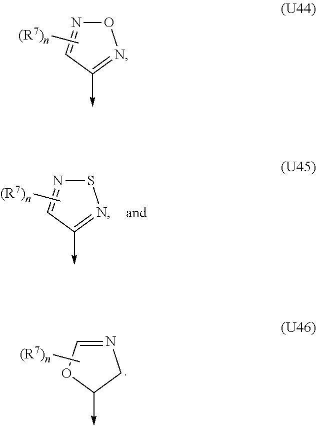
wherein
R⁷ is as defined in claim 1;
R^{7a} is selected from the group consisting of hydrogen, C₁-C₄alkyl, C₁-C₄haloalkyl and C₃-C₄cycloalkyl; and
n=0, 1 or 2.

3. A compound according to claim 2, wherein U is selected from the group consisting of U13, U23, U25, U27 and U28.

4. A compound according to claim 1, wherein Q is selected from the group consisting of Q 1 to Q-11: 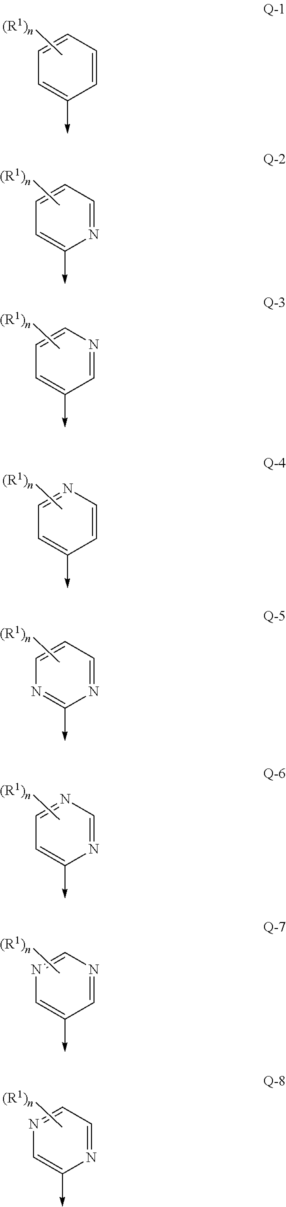 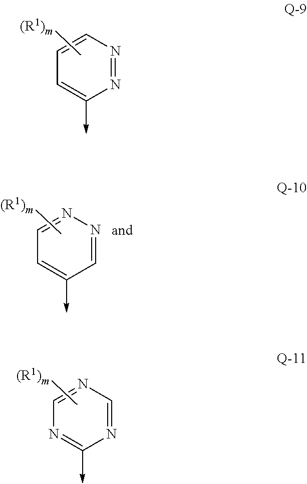
wherein R¹ is as defined in claim 1; and
m is 0, 1 or 2.

5. A compound according to claim 4, wherein Q is selected from the group consisting of Q-1, Q-3 and Q-7.

6. A compound according to claim 7, wherein Q is Q-1.

7. A compound according to claim 5, wherein m is 1.

8. A compound according to claim 1, wherein R² is halogen or C₁-C₄haloalkyl.

9. A compound according to claim 8, wherein R² is —CF₃ or —CF₂H.

10. A compound according to claim 1, wherein R³ is hydrogen or halogen.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling weeds at a locus comprising application to the locus of a weed controlling amount of a composition according to claim 11.

15. Use of a compound of Formula (I) as defined in claim 1 as a herbicide.
